(19) United States
(12) Patent Application Publication
(10) Pub. No.: US 2015/0104470 A1
(43) Pub. Date: Apr. 16, 2015

(54) IMMUNE MODULATION BY PERI-LYMPHATIC OR INTRA-LYMPHATIC CELL THERAPY

(71) Applicant: Neil H. Riordan, Trophy Club, TX (US)

(72) Inventor: Neil H. Riordan, Trophy Club, TX (US)

(21) Appl. No.: 14/512,285

(22) Filed: Oct. 10, 2014

Related U.S. Application Data
(60) Provisional application No. 61/890,170, filed on Oct. 11, 2013.

(51) Int. Cl. A61K 35/28; A61K 35/54; A61K 35/48; A61K 35/14

(52) U.S. Cl. CPC A61K 35/28; A61K 35/17; A61K 35/15; A61K 35/545; A61K 35/48
USPC 424/184.1

(57) ABSTRACT

Disclosed are compositions of matter, methods of treatment, and protocols useful for therapeutic immune modulation using cell therapy administered perilymphatically or intralymphatically. In one particular embodiment, the invention provides means of treating an autoimmune condition by perilymphatic administration of a mesenchymal stem cell population. Said mesenchymal stem cell populations may be derived from umbilical cord tissues such as the Wharton's Jelly, amniotic membranes, or amniotic stem cells. In another particular embodiment Sertoli cells may be utilized as immune modulatory cells for the practice of the invention.

### CROSS-REFERENCE TO RELATED APPLICATIONS

This application claims priority to and is a non-provisional of co-pending U.S. Provisional Application Ser. No. 61/890,170, filed Oct. 11, 2013, entitled “Immune Modulation by Peri-lymphatic or Intra-lymphatic Cell Therapy”, which is expressly incorporated herein by reference in its entirety.

### FIELD OF THE INVENTION

The invention pertains to the field of immune modulation, more specifically, the invention pertains to the field of administration of immune modulatory cells and agents, more specifically, the invention pertains to novel methods of treatment and protocols for augmenting immune modulation in a manner suppressive to disorders associated with pathological immune responses.

### BACKGROUND

The basic concept of cellular therapy has been known since the time of Paracelsus, who in the 16th Century stated “Heart heals the heart, lung heals lung, spleen heals spleen; like cures like.” These philosophical ponderings of this alchemist were reduced to practice by the controversial Swiss physician Paul Niehans who utilized fetal xenogeneic cells to treat a variety of ailments in the early part of the last century. In recent times cell therapy has been gaining momentum for treatment of a wide variety of disorders. By far the most widely established use of cell therapy is for treatment of leukemias in the form of bone marrow transplantation.

The first hematopoietic stem cell transplant, or bone marrow transplant, was performed in 1956 by Dr. E. Donnall Thomas using bone marrow cells isolated from an identical twin donor for a recipient who had leukemia. The idea was that if the patient was irradiated with high doses, then the radiation would kill all of the leukemia cells. Unfortunately, the radiation would also destroy the healthy bone marrow stem cells. So the idea was to utilize donor bone marrow to replenish the recipient with healthy hematopoietic stem cells. Dr. Thomas, along with Joseph E. Murray, won the Nobel Prize in 1990 for this discovery.

As described above, transfer of bone marrow stem cells has been performed for decades. Scientists have postulated, whether the bone marrow stem cell possesses the potential to differentiate into all the different types of blood cells, maybe it can also differentiate into other cells as well. This process was originally termed “transdifferentiation”. The first report of transdifferentiation to appear in the major medical literature was a paper by Orlic et al. [1], in which mouse bone marrow derived stem cells were injected into mice that were given an experimental heart attack. The interesting thing about this experiment was that the bone marrow stem cells used were labeled to glow green. The donor animals were genetically engineered to express the green fluorescent protein (GFP) gene throughout their bodies. This essentially means that all cells derived from the GFP donor mice were green. Additionally, the experimenters purified the mouse equivalent of the human CD34 bone marrow hematopoietic stem cell. The molecular markers used where positivity for stem cell antigen (SCA-1) and negativity for the lineage markers (lin negative). Following induction of a heart attack by ligation of one of the coronary arteries, the researchers implanted the cells in the area of infarct. The mice which received implanted hematopoietic stem cells, but not control cells, had increased pumping ability of the heart and decreased levels of heart damage.

Numerous other experiments have demonstrated efficacy of cell therapy in animals and humans for non-hematopoietic purposes. For example Japanese researchers have demonstrated that when bone marrow cells are injected into the heart muscle of patients undergoing bypass surgery a therapeutic effect is observed. The idea was that the injected bone marrow cells will stimulate production of new blood vessels and thereby increase oxygenation to the heart [2]. The procedure, although highly invasive, was associated with no treatment related adverse effects and 3 out of the 5 patients had increased blood vessel production as assessed radiologically, as well as improved cardiac function. This first demonstration in 2001, was repeated by numerous investigators. In 2003, the study was repeated using CD133 purified bone marrow stem cells and published in the prestigious journal Lancet [3], reporting positive results. Subsequently numerous studies have been conducted in the area of cardiology demonstrating that administration of a patient's own bone marrow is associated with positive outcome. Another example of cell therapy was a program conducted by Layton Biosciences, who developed a homogeneous cellular population by differentiating a proprietary teratocarcinoma cell line into neurons using a retinoic acid based protocol. These cells, called LBS-neurons were utilized in several clinical trials. In one trial surgical implantation of these cells was demonstrated to induce improvement based on the functional ESS score in some patients [4].

Cell therapy has also been used in the treatment of diabetes, for example, the Edmonton Protocol involves intrahepatic administration of donor islets under the cover of calcineurin-sparing immune suppressants. This approach has resulted in reduced insulin requirements of Type I diabetics, and in some cases achievement of complete insulin independence [5,6]. Other uses of cell therapy include treatment of stroke [7], liver failure [8], lung failure [9], and peripheral artery disease [10]. Unfortunately, in comparison with other indications, the use of cellular therapy for treatment of autoimmunity has been relatively underdeveloped.

The specific invention describes the possibility of utilizing cell therapy for the purpose of immune modulation, more specifically, involving a novel method of administering cell therapy and novel protocols.

There exists in the art a need for modulating the immune system in patients with autoimmune conditions. Autoimmune diseases affect approximately 5-8 percent of the American population, being the 3rd major cause of illness behind cancer and heart disease. Autoimmune diseases are conditions in which the body's immune response initiates immunological attacks against tissue belonging to “self”. Widely known autoimmune conditions include type 1 diabetes, in which the body attacks the insulin producing beta cells, multiple sclerosis, in which the myelin basic protein in the myelin sheaths is destroyed, rheumatoid arthritis, in which T cells orchestrate damage to the synovial tissue of the joints, and lupus, in which anti-nuclear antibodies cause systemic inflammation and complement activation.

The concept of specifically coaxing the immune system to stop attacking certain antigens, while allowing immunity to other antigens to remain intact is termed “immunological tolerance”. The concept of “immunological tolerance” dates back to the days of Medawar and observations that shared circulation during fetal development leads to selective immunological nonresponsiveness to genetically discordant fraternal not third party [11]. The word “tolerance” can mean numerous states and can be achieved by numerous pathways. Tolerance in its full sense requires lack of immunological attack on the target antigen or tissue. There are two general, non-mutually exclusive, means in which this occurs: Stimulation of Treg cells that actively suppress responses to the specific antigen; or clonally inactivating the T cells that are responding to the specific antigen. However, in order to achieve a therapeutic response in a disease condition it is not strictly necessary to achieve “full tolerance” but in some situations immune modulation may be sufficient. For example, inhibition of Th17 responses or deviation from Th17 to Th2/Th3 may be sufficient to elicit a clinical effect. Although not strictly correct, for the purposes of this discussion, we will use the word “tolerance” to include immune deviation.

Tolerance naturally occurs in several situations such as pregnancy, cancer, following ingestion of antigen, or administration of antigen into the anterior chamber of the eye. In animal studies, immune deviation in pregnancy was demonstrated by observations of selective immunological non-responsiveness in T cells recognizing fetally-expressed antigens [1,2]. Clinically, it is believed that a substantial number of pregnancy failures in the first trimester are associated with immunological causes [1,3]. In neoplasia, transgenic expression of defined antigens on tumors leads to selective inhibition of systemic T cell responses to the specific antigens [14-16]. The ability of tumors to inhibit peripheral T cell activity has been associated in numerous studies with poor prognosis [17-19]. Ingestion of antigen, including the RA autoantigen collagen II [20], has been shown to induce inhibition of both T and B cell responses in a specific manner [21, 22]. Remission of disease in animal models of RA [23], multiple sclerosis [24], and type I diabetes [25], has been reported by oral administration of autoantigens. Anterior chamber associated immune deviation (ACAID) is a phenomena in which local implantation of antigen results in a systemic immune modulation towards the antigen. Commonly this is demonstrated by antigen-specific suppression of DTH responses after intra-chamber administration of antigen [26]. Induction of ACAID has been used therapeutically in treatment of a mouse model of pulmonary inflammation: pretreatment with anterior chamber antigen injection resulted in systemic production from pulmonary damage [27].

All of these situations of natural immune deviation have certain common cellular processes: a) specialized antigen presenting cells; b) induction T cells with regulatory activity; and c) deviation of cytokine production and/or suppression of effector cell activity.

To provide specific background, we will discuss the example of multiple sclerosis (MS) in which immunological damage can be observed by MRI in the form of plaques, and functionally is manifested by the patient experiencing the various characteristics of MS ranging from visual (e.g. optic neuritis, nystagmus, etc), motor (e.g. paresis, spasticity, etc), sensory (e.g. paraesthesia), balance (e.g. ataxia, vertigo) and cognitive (depression, cognitive dysfunction) alterations [28].

There are 4 main types of MS: 1) Relapse-remitting MS is the condition which the majority (about 85-90%) of MS patients are initially diagnosed with. As the name indicates, this type is characterized by relapses followed by periods of remission in which disease activity subsides. It is believed that during remission the oligodendrocytes “fix” the neurons by producing new myelin; 2) Secondary progressive MS usually occurs as a progression of the relapse remitting type at the point where remissions decrease in frequency and eventually the debilitating characteristics continually progress. On average it takes about 19 years for MS to convert from relapse-remitting to secondary progressive; 3) Primary progressive is characterized by patients presenting with MS in which no remissions are seen; and 4) In the progressive relapsing form, a continuous increase in symptoms is seen, however spikes of accelerated disease activity are interspersed in the progression of the condition [29].

Depending on the type of MS, various treatments are routinely used. These include steroids, immune suppressants (cyclosporine, azathioprine, methotrexate), immune modulators (interferons, glatiramer acetate), and immune modulating antibodies (natalizumab) [30]. The general therapeutic approach is to rapidly treat relapses so as to minimize permanent damage, as well as to prevent onset of relapse or progression to more advanced forms of MS. Unfortunately long term efficacy data is not available for many of the current approaches used clinically. At present none of the MS treatment available on the market selectively inhibit the immune attack against the nervous system, nor do they stimulate regeneration of previously damaged tissue. Experimental approaches in clinical trials using peptide/protein vaccines to antigen-specifically inhibit immune responses, however even if successful this approach will not induce regeneration [31-34]. Other experimental approaches include the use of bone marrow stem cells in combination with lymphodepletion to destroy the original immune system of the patient and subsequently attempt to “reset it” [35-37]. Although stem cells are used in this approach, again there is little evidence of active regeneration.

An ideal approach to MS would address the problems of: a) reversing the misdirected immune attack and b) stimulating regeneration of damage that has already been caused. Mesenchymal stem cells (MSC) have been demonstrated in numerous animal models of multiple sclerosis [38-41] and pilot clinical investigations [42, 43] to inhibit pathological immune responses while stimulating regeneration of damaged nerve tissue. Unfortunately MSC have limitations in terms of production, costs, ability to immune modulate, and therapeutic efficacy. In the current invention, mesenchymal stem cells administered intralymphatically or perilymphatically are utilized as a novel cellular approach towards treatment of MS.

Rheumatoid Arthritis (RA) is a chronic autoimmune condition characterized by non-specific, usually symmetric inflammation of the peripheral joints, potentially resulting in progressive destruction of articular and periarticular structures, with or without generalized manifestations. Although its precise etiology has not yet been determined, genetic predisposition is well documented. In addition, environmental factors are thought to play a role. According to the American College of Rheumatology (1987), at least four of the following criteria have to be met before a condition is classified as rheumatoid arthritis: 1) morning stiffness of >1 hour most mornings for at least 6 weeks; 2) arthritis and soft-tissue swelling of >3 of 14 joints/joint groups, present for at least 6 weeks; 3) arthritis of hand joints, present for at least 6 weeks; 4) symmetric arthritis, present for at least 6 weeks; 5) subcutaneous nodules in specific places; 6) rheumatoid factor at a level above the 95th percentile; and 7) radiological changes suggestive of joint erosion.

In RA, the synovial tissue becomes markedly thickened and swollen. As the disease progresses, there is gradual proliferation and recruitment of synoviocytes, as well as inflammatory cells into the synovium [44, 45]. Up to 50% of the infiltrating leukocytes in the synovium are T-lymphocytes, primarily CD4+ T cells, with an activated/memory phenotype [46-48], with some investigators reporting a Th1 bias [48, 49], and others reporting a Th17 bias [50-54]. Cells of monocyte/macrophage origin also become prominent in the rheumatoid synovium, accounting for up to 20% of cells, and they too exhibit an activated phenotype. Monocyte/macrophage-like cells in the rheumatoid synovium produce an array of proinflammatory molecules, including the cytokines IL-1, TNF-.alpha., IL-6, GM-CSF as well as proteolytic enzymes including collagenases and matrix metalloproteinases. Monocytes from RA patients have been demonstrated to elicit recruitment of Th17 cells [55]. B-cells, plasma cells and neutrophils account for less than 5% of cells in the rheumatoid synovium, although neutrophils are prominent in the synovial fluid. Interestingly, neutrophils have been demonstrated to augment chemotaxis and activation of Th17 cells [56]. As synovial proliferation and inflammation advances, the expanding mass of vascular, inflammatory synovial tissue is termed as the pannus. Pannus is responsible for invading articular cartilage and destroying bone. The products of activated T cells are felt to be the driving factors behind the formation and expansion of pannus.

Conventionally, RA treatment involves initiating Disease Modifying Anti-Rheumatic Drug (DMARD) therapy following diagnosis with subsequent optimization of drug therapy in order to have a greater beneficial impact on disease outcome [57]. DMARDs are classified as immunomodulators and immunosuppressants on the basis of their action mechanism. Two of the commonly used DMARDS are methotrexate (N-[4-[(2,4-diamino-6-pteridinyl)methylamino]benzoyl]-L-glutamic acid), an immunosuppressant that antagonizes folic acid metabolism and Leflunomide (N-(4-trifluoromethylphenyl)-5-methylisoxazole-4-carboxamide) which is an inhibitor of pyrimidine biosynthesis inhibitory action However, since both methotrexate and leflunomide can induce serious side effects including infection or interstitial pneumonia, their dose of administration must be tightly monitored. Other DMARDS include gold compounds, hydroxychloroquine, sulfasalazine, combinations of slow-acting drugs, corticosteroids, and cytotoxic or immunosuppressive drugs.

Aplastic anemia is characterized by lack of hematopoiesis with strong evidence that in some patients an autoimmune component exists. Specifically, it is a disease of the bone marrow. The bone marrow stops making enough red blood cells, white blood cells and platelets for the body. Any blood cells the marrow does make are normal, but there are not enough of them. Aplastic anemia can be moderate, severe or very severe. People with severe or very severe aplastic anemia are at risk for life-threatening infections or bleeding. While patients with moderate AA often respond to immune suppressive agents, currently there are no treatment options for patients with severe AA who are lacking a suitable bone marrow donor. It is known that patients with aplastic anemia have a deficiency in numbers of T regulatory cells, as well as enhanced activity of Th17 cells. In one study, Kordasti et al. investigated 63 patients with acquired AA. Th1 and Th2 cells were significantly higher in AA patients than in healthy donors. Tregs were significantly lower in patients with severe AA than in healthy donors and patients with non-severe AA. Th17 cells were increased in severe AA but normal in non-severe AA. Activated and resting Tregs were reduced in AA, whereas cytokine-secreting non-Tregs were increased. Tregs from AA patients were unable to suppress normal effector T cells. In contrast, AA effector T cells were suppressible by Tregs from healthy donors. Th1 clonality in AA, investigated by high-throughput sequencing, was greater than in healthy donors.

Diabetes mellitus is a metabolic disorder that occurs in approximately four percent of humans. There are two types of diabetes; the non-insulin-dependent or “maturity onset” form (Type 2) and the insulin-dependent or “juvenile onset” form (Type 1). Clinically, the majority of Type 2 diabetics are obese, with manifestations of clinical symptoms of the disease usually appearing in patients over age 40. In contrast, Type 1 diabetics are usually not over-weight relative to their age and height and typically exhibit rapid onset of the disease at an early age, often before age 30.

One-third of diabetes patients suffer from Type 1 diabetes (Foster et al., Harrison's Principles of Internal Medicine, Chap. 114, pp. 661-678, 10th Ed., McGraw-Hill, New York). Type 1 diabetes is an autoimmune disease wherein a state of hyperglycemia results from the T-cell mediated destruction of insulin-secreting b-cells in the pancreatic Islets of Langerhans (Eisenbarth et al., 1986, New Engl. J. Med. 314: 1360-1368). In the pancreas, the islets of Langerhans contain several cell types that secrete distinct hormones. Each cell type expresses different tissue-specific proteins: a cells express glucagon; .beta. cells express insulin; and, .delta. cells express somatostatin. In insulin-dependent diabetes an effector T cell recognizes peptides from a .beta. cell-specific protein and kills the .beta. cells. Glucagon and somatostatin are still produced by the .alpha. and .delta. cells, but no insulin can be made. The disease manifests itself as a series of hormone-induced metabolic abnormalities which eventually lead to serious, long-term and debilitating complications involving several organ systems including the eyes, kidneys, nerves, and blood vessels. Pathologically, the disease is characterized by lesions of the basement membranes, demonstrable under electron microscopy. Type 1 diabetics characteristically show very low or immeasurable plasma insulin with elevated glucagon. Regardless of what the exact etiology is, most Type 1 patients have circulating antibodies directed against their own pancreatic cells including antibodies to insulin, to the islet of Langerhans cell cytoplasm and to the enzyme glutamic acid decarboxylase. An immune response specifically directed against beta cells (insulin producing cells) leads to Type 1 diabetes. Current therapeutic regimens for Type 1 diabetes include modifications to the diet to minimize hyperglycemia resulting from the lack of natural insulin, which in turn, is the result of damaged beta cells. Diet is also modified with regard to insulin administration to counter the hypoglycemic effects of the hormone. Whatever the form of treatment, parenteral administration of insulin is required for all Type 1 diabetics, hence the term “insulin-dependent” diabetes. Because Type 1 diabetes usually manifests itself in adolescents and because the subcutaneous delivery of insulin requires strict self-regimentation, compliance is often a serious problem. For the clinician, it is difficult to precisely regulate the amounts of insulin needed at any given time of the patient's day. Furthermore, it is all but impossible to regulate blood glucose levels in diabetic patients with parenteral insulin to the extent to which blood glucose is regulated in normal individuals. Thus, in the early stages of treatment of Type 1 diabetes, patients often become either hyperglycemic or hypoglycemic because the exact timing of the insulin injections and levels of insulin needed are not known. As treatment progresses the clinician and, more importantly, the patient adjusts to the daily routine, but there is always the risk of ketoacidosis or hypoglycemia.

Previous means of immune modulation have been described in the literature, specifically, U.S. Pat. No. 6,277,635 relates to the use of IL-10 for suppressing transplant rejection. This patent teaches methods of treating and inhibiting tissue rejection, inhibiting GVHD and antigen specific responses. It further describes T cells that exhibit anergy for a particular antigen. U.S. Pat. No. 6,428,985 describes mammalian, including human, immunosuppressive compositions containing IL-10 polypeptides with at least one mutation in the native sequence (Mut IL-10), either alone or in combination with other agents, and various in vitro and in vivo methods of using such compositions and combinations thereof. Uses include immunosuppressive and combination therapies for a number of diseases and disorders related to inflammation, transplantation, fibrosis, scarring, and tumor treatment. The effect of Mut IL-10 has been shown in animal studies but not in human clinical settings. U.S. Pat. No. 6,022,536 describes the combined use of IL-10 and cyclosporine as immunosuppression therapy for treating autoimmune diseases and GVHD. Synergistic combination of low doses of IL-10 and cyclosporine and a pharmaceutical carrier are proposed. U.S. Pat. No. 6,403,562 describes methods for treating autoimmune-related diseases, such as multiple sclerosis, by administering IL-10 together with TGF-.beta., to a person afflicted with or predisposed to an autoimmune disease. These cytokines act in a synergistic manner as suppressor factors to inhibit the activation of self-reactive T cells that are involved in autoimmune disease.

### SUMMARY

The invention provides novel treatment method, protocols, and compositions of matter for treatment of mammals suffering from disorders of immune dysregulation. In aspect, the invention provides treatments for autoimmune disorders, in another aspect, the invention provides means of inducing immunological tolerance to either an autoantigen, an alloantigen, or a xenoantigen. In one aspect the invention provides means of preventing rejection of a transplanted organ, or cellular graft. In one aspect the invention provides means of treating graft versus host conditions.

One central aspect of the invention is the finding that administration of immune modulatory cells, with specific examples of mesenchymal stem cells, amniotic membrane derived stem cells, or Sertoli cells via the perilymphatic, and/or intralymphatic routes induces a potent immune modulatory response that is capable of inducing remission in patients with autoimmune conditions. Based on this finding, one of skill in the art is thought to apply this finding to utilization of other immune modulatory cells, as well as in combination with other means of augmenting the process of immunological tolerogenesis.

Methods herein include embodiments wherein one or more cells are co-administered to said recipient based on specific need for immune modulation in said recipient. Methods wherein an antigen is administered in combination with said immune modulatory cells. Methods wherein an antigen is used to pulse said dendritic cell or CD5 positive B cell prior to administration of said cell. Methods wherein said antigen is selected from a group comprising of: a) a protein; b) a peptide; and c) an altered peptide ligand. Methods wherein perilymphatic administration is performed by a subcutaneous injection proximal to an area that drains into one or a plurality of lymph nodes. Methods wherein said perilymphatic administration is performed by a intradermal injection proximal to an area that drains into one or a plurality of lymph nodes. Methods wherein said area that drains into one or a plurality of lymph nodes is identified by manual means. Methods wherein said area that drains into one or a plurality of lymph nodes is identified by a visualization means. Methods wherein said visualization means is comprised of lymphangiography. Methods wherein perilymphatic administration is considered intralymphatic administration. Methods wherein said cells possessing immune modulatory activity are administered at a concentration ranging from 1,000 cells to 100 million cells. Methods wherein said cells possessing immune modulatory activity are administered at a concentration ranging from 10,000 cells to 10 million cells. Methods wherein said cells possessing immune modulatory activity are administered at a concentration of approximately 100,000 cells. Methods wherein said cells possessing immune modulatory activity are administered into a superficial inguinal lymph node asceptically.

Methods wherein said cells possessing immune modulatory activity are administered in a volume of approximately 0.1 ml. Methods wherein said cells possessing immune modulatory activity are administered slowly under ultrasound guidance. Methods wherein small aspiration of injection site is first performed prior to intra or perilymphatic administration of said cells possessing immune modulatory activity, so as to avoid inadvertent intravascular administration. Methods wherein said cells possessing immune modulatory properties are administered into several sites intralymphatically or perilymphatically. Methods wherein said administration is performed according to a frequency of administration required to evoke a desired therapeutic response. Methods wherein said administration is performed on a daily basis. Methods wherein said administration is performed every second day. Methods wherein said administration is performed on a weekly basis. Methods wherein said cells possessing immune modulatory activity are administered by injection into a dorsal pedal lymphatic channel. Methods wherein said injection into a dorsal pedal lymphatic channel is isolated after Evans blue is infiltrated and lidocaine hydrochloride is administered for local anesthesia according to standard methods used for lymphangiography.

Methods of treating an autoimmune condition in a mammal through administration of an immune modulatory cell perilymphatically or intralymphatically. Methods wherein said autoimmune condition is selected from a group comprising of: a) systemic lupus erythromatosus, b) multiple sclerosis; c) type 1 diabetes, d) rheumatoid arthritis, e) myasthenia gravis, f) scleroderma, g) psoriasis, and h) autoimmune cardiomyopathy. Methods wherein said autoimmune condition is defined as an upregulation of Type 1 immunity in an individual compared to the baseline in a representative, age-matched, healthy control population. Methods wherein said autoimmune condition is defined as an upregulation of Type 17 immunity in an individual compared to the baseline in a representative, age-matched, healthy control population. Methods wherein said autoimmune condition is defined as a dysfunction of T regulatory cells in an individual compared to the baseline in a representative, age-matched, healthy control population. Methods wherein said dysfunction of T regulatory cells is characterized as a decreased ability of said T regulatory cells to inhibit proliferation of a conventional T cell after antigenic stimulation of said conventional T cell. Methods wherein said dysfunction of T regulatory cells is characterized as a decreased ability of said T regulatory cells to inhibit cytokine production of a conventional T cell after antigenic stimulation of said conventional T cell.

Methods wherein autoimmune condition is defined as an upregulation of natural killer cell activity in an individual compared to the baseline in a representative, age-matched, healthy control population. Methods wherein said natural killer cell activity is by ability of said natural killer T cells to induce lysis in a target cell susceptible to natural killer cell mediated lysis. Methods wherein said target cell is selected from a group comprising of: a) K562; b) YAC; and c) Jurkat. Methods wherein said immune modulatory cell is selected from a group of cells comprising of: a) mesenchymal stem cells; b) T regulatory cells; c) type 2 monocytes; d) CD5 positive B cells; e) type 2 NKT cells; f) tolerogenic dendritic cells; g) gamma delta T cells; h) T cells with immune regulatory properties; i) CD34 cells; j) very small embryonic like stem cells and k) Sertoli cells. Methods wherein said mesenchymal stem cell is derived from tissue comprising a group selected from: a) Wharton's Jelly; b) bone marrow; c) peripheral blood; d) mobilized peripheral blood; e) endometrium; 0 hair follicle; g) deciduous tooth; h) testicle; i) adipose tissue; j) skin; k) amniotic fluid; 1) cord blood; m) omentum; n) muscle; o) amniotic membrane; and o) periventricular fluid.

Methods wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Methods wherein said mesenchymal stem cells do not express substantial levels of HLA-DR, CD117, and CD45. Methods wherein said mesenchymal stem cells are generated from a pluripotent stem cell. Methods wherein said pluripotent stem cell is selected from a group comprising of: a) an embryonic stem cell; b) an inducible pluripotent stem cell; c) a parthenogenic stem cell; and d) a somatic cell nuclear transfer derived stem cell. Methods wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Methods wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging. Methods wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81. Methods wherein said somatic cell nuclear transfer derived stem cells possess a phenotype negative for SSEA-1 and positive for SSEA-3, SSEA-4, TRA-1-60, TRA-1-81, and alkaline phosphatase. Methods wherein said mesenchymal stem cells are differentiated from a pluripotent stem cell source through culture in the presence of an inhibitor of the SMAD-2/3 pathway. Methods wherein said mesenchymal stem cells are differentiated from a pluripotent stem cell source through culture in the presence of an inhibitor nucleic acid targeting the SMAD-2/3 pathway. Methods wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme. Methods wherein said inhibitor of the SMAD-2/3 pathway is a small molecule inhibitor. Methods wherein said small molecule inhibitor is SB-431542. Methods wherein a selection process is used to enrich for mesenchymal stem cells differentiated from said pluripotent stem cell population. Methods wherein said enrichment method comprises of positively selecting for cells expressing a marker associated with mesenchymal stem cells.

Methods wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Methods, wherein said immune modulatory cells are autologous, allogeneic or xenogeneic to the recipient. Methods wherein one or more cells are co-administered to said recipient based on specific need for immune modulation in said recipient. Methods wherein an antigen is administered in combination with said immune modulatory cells. Methods wherein an antigen is used to pulse said dendritic cell or CD5 positive B cell prior to administration of said cell. Methods wherein said antigen is selected from a group comprising of: a) a protein; b) a peptide; and c) an altered peptide ligand. Methods wherein perilymphatic administration is performed by a subcutaneous injection proximal to an area that drains into one or a plurality of lymph nodes. Methods wherein said perilymphatic administration is performed by a intradermal injection proximal to an area that drains into one or a plurality of lymph nodes. Methods wherein said area that drains into one or a plurality of lymph nodes is identified by manual means. Methods wherein said area that drains into one or a plurality of lymph nodes is identified by a visualization means. Methods wherein said visualization means is comprised of lymphangiography. Methods wherein perilymphatic administration is considered intralymphatic administration. Methods wherein said cells possessing immune modulatory activity are administered at a concentration ranging from 1,000 cells to 100 million cells. Methods wherein said cells possessing immune modulatory activity are administered at a concentration ranging from 10,000 cells to 10 million cells. Methods wherein said cells possessing immune modulatory activity are administered at a concentration of approximately 100,000 cells. Methods wherein said cells possessing immune modulatory activity are administered into a superficial inguinal lymph node asceptically. Methods wherein said cells possessing immune modulatory activity are administered in a volume of approximately 0.1 ml. Methods wherein said cells possessing immune modulatory activity are administered slowly under ultrasound guidance. Methods wherein small aspiration of injection site is first performed prior to intra or perilymphatic administration of said cells possessing immune modulatory activity, so as to avoid inadvertent intravascular administration. Methods wherein said cells possessing immune modulatory properties are administered into several sites intralymphatically or perilymphatically. Methods wherein said administration is performed according to a frequency of administration required to evoke a desired therapeutic response. Methods wherein said administration is performed on a daily basis. Methods wherein said administration is performed every second day. Methods wherein said administration is performed on a weekly basis. Methods wherein said cells possessing immune modulatory activity are administered by injection into a dorsal pedal lymphatic channel. Methods wherein said injection into a dorsal pedal lymphatic channel is isolated after Evans blue is infiltrated and lidocaine hydrochloride is administered for local anesthesia according to standard methods used for lymphangiography.

A method of treating multiple sclerosis in mammal through administration of a mesenchymal stem cell population perilymphatically or intralymphatically. Methods wherein at least 50% of said mesenchymal stem cells are positive for a marker selected from a group comprising of: CD10, CD24, IL-11, AIRE-1, ANG-1, CXCL1, CD105, CD73 and CD90. Methods wherein at least 50% of said mesenchymal stem cells are positive for a marker selected from a group comprising of: CD10, CD24, IL-11, AIRE-1, ANG-1, CXCL1, CD73, CD90, CD105, CD13, CD29, CD 44, CD166, and CD274. Methods wherein at least 50% of said mesenchymal stem cells do not express or express low levels markers selected from a group comprising of: CD14, CD31, CD34, CD45, CD133, FGFR2, and CD271. Methods wherein said mesenchymal stem cells are concentrated from a tissue population by means of binding to a molecule possessing a preferential adherence for said mesenchymal stem cell compared to other cells in said tissue population.

Methods wherein said molecule possessing a preferential adherence for said mesenchymal stem cell compared to other cells in said tissue population is selected from a group comprising of: antibodies, microbodies, aptamers, peptides, and proteins. Methods wherein said molecules possess affinity towards markers selected from a group comprising of CD10, CD24, IL-11, AIRE-1, ANG-1, CXCL1, CD73, CD90, CD105, CD13, CD29, CD 44, CD166, and CD274. Methods wherein said peptides are selected from the group consisting of tenascin, collagen-1, collagen-3, collagen-4, thrombospondin-1, osteopontin, fibronectin, vitronectin, and mixtures thereof, thereby allowing binding of the peptide or fragment thereof to mesenchymal stem cells, selecting and/or enriching the mesenchymal stem cells bound to the peptide or fragment thereof from the other cell types not bound to the peptide or fragment thereof. Methods wherein said mesenchymal stem cells are derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; and o) periventricular fluid. Methods wherein said mesenchymal stem cells are generated from a pluripotent stem cell. Methods wherein said pluripotent stem cell is selected from a group comprising of: a) an embryonic stem cell; b) an inducible pluripotent stem cell; c) a parthenogenic stem cell; and d) a somatic cell nuclear transfer derived stem cell. Methods wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT). Methods wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging. Methods wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81. Methods wherein said somatic cell nuclear transfer derived stem cells possess a phenotype negative for SSEA-1 and positive for SSEA-3, SSEA-4, TRA-1-60, TRA-1-81, and alkaline phosphatase. Methods wherein said mesenchymal stem cells are differentiated from a pluripotent stem cell source through culture in the presence of an inhibitor of the SMAD-2/3 pathway. Methods wherein said mesenchymal stem cells are differentiated from a pluripotent stem cell source through culture in the presence of an inhibitor nucleic acid targeting the SMAD-2/3 pathway. Methods wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme. Methods wherein said inhibitor of the SMAD-2/3 pathway is a small molecule inhibitor. Methods wherein said small molecule inhibitor is SB-431542. Methods wherein said immune modulatory cells are autologous, allogeneic or xenogeneic to the recipient. Methods wherein one or more cells are co-administered to said recipient based on specific need for immune modulation in said recipient. Methods wherein an antigen is administered in combination with said immune modulatory cells. Methods wherein an antigen is used to pulse said dendritic cell or CD5 positive B cell prior to administration of said cell. Methods wherein said antigen is selected from a group comprising of: a) a protein; b) a peptide; and c) an altered peptide ligand. Methods wherein perilymphatic administration is performed by a subcutaneous injection proximal to an area that drains into one or a plurality of lymph nodes. Methods wherein said perilymphatic administration is performed by a intradermal injection proximal to an area that drains into one or a plurality of lymph nodes. Methods wherein said area that drains into one or a plurality of lymph nodes is identified by manual means. Methods wherein said area that drains into one or a plurality of lymph nodes is identified by a visualization means. Methods wherein said visualization means is comprised of lymphangiography. Methods wherein perilymphatic administration is considered intralymphatic administration. Methods wherein said cells possessing immune modulatory activity are administered at a concentration ranging from 1,000 cells to 100 million cells. Methods wherein said cells possessing immune modulatory activity are administered at a concentration ranging from 10,000 cells to 10 million cells. Methods wherein said cells possessing immune modulatory activity are administered at a concentration of approximately 100,000 cells. Methods wherein said cells possessing immune modulatory activity are administered into a superficial inguinal lymph node asceptically. Methods wherein said cells possessing immune modulatory activity are administered in a volume of approximately 0.1 ml. Methods wherein said cells possessing immune modulatory activity are administered slowly under ultrasound guidance. Methods wherein small aspiration of injection site is first performed prior to intra or perilymphatic administration of said cells possessing immune modulatory activity, so as to avoid inadvertent intravascular administration. Methods wherein said cells possessing immune modulatory properties are administered into several sites intralymphatically or perilymphatically. Methods wherein said administration is performed according to a frequency of administration required to evoke a desired therapeutic response. Methods wherein said administration is performed on a daily basis. Methods wherein said administration is performed every second day. Methods wherein said administration is performed on a weekly basis. Methods wherein said cells possessing immune modulatory activity are administered by injection into a dorsal pedal lymphatic channel. Methods wherein said injection into a dorsal pedal lymphatic channel is isolated after Evans blue is infiltrated and lidocaine hydrochloride is administered for local anesthesia according to standard methods used for lymphangiography.

Methods of immune modulating a mammal through administration of a conditioned media from an immune modulatory cell, said conditioned media administered perilymphatically or intralymphatically. Methods wherein said conditioned media is obtained by culturing a viable cell population under conditions that are physiological or near-physiological. Methods wherein said conditioned media is obtained by culturing a viable cell population under conditions that are non-physiological. Methods wherein said conditioned media of a cultured cell population is substantially free of cellular debris. Methods wherein said cultured cells are exposed to conditions selected from a group comprising of: a) exposure to hypoxia; b) treatment with a histone deacetylase inhibitor; c) treatment with a growth factor; d) treatment with a DNA methyltransferase inhibitor; and e) exposure to hyperthermia. Methods wherein said growth factor is selected from a group comprising of: a WNT signaling agonist, TGF-b, bFGF, IL-6, SCF, BMP-2, thrombopoietin, EPO, IGF-1, IL-11, IL-5, Flt-3/Flk-2 ligand, fibronectin, LIF, HGF, NFG, angiopoietin-like 2 and 3, G-CSF, GM-CSF, Tpo, Shh, Wnt-3a, Kirre, and a mixture thereof. Methods wherein said conditioned media is generated by contacting cells with a liquid media, wherein said liquid media is selected from a group of media useful for maintaining cell viability in culture consisting of a group comprising of: a) alpha MEM; b) DMEM; c) RPMI; d) Opti-MEM; e) IMEM; and f) AIM-V media.

Methods wherein said cell population is in contact with a liquid media, wherein said cells are expanded in liquid media containing fetal calf serum and subsequently cultured in media substantially lacking said fetal calf serum, with said culture lacking fetal calf serum used for production of a therapeutic product. Methods wherein said cell population is cultured in a serum-free media. Methods wherein said cell population is in contact with a liquid media, wherein said contact between said cell and liquid media is between 1 minute to 96 hours. Methods wherein said cell population is in contact with a liquid media, wherein said contact between said cell and liquid media is between 12 hours to 72 hours. Methods wherein said cell population is in contact with a liquid media, wherein said contact between said cell and liquid media is between 24 hours to 48 hours. Methods wherein said cell population is in contact with a liquid media, said contact between said cell and liquid media is approximately 24 hours. Methods wherein said cell population is in contact with a liquid media, wherein said contact between said cell and liquid media is selected for a timepoint in which optimal secretion of therapeutic factors occurs in said liquid media. Methods wherein said immune modulatory cell is selected from a group of cells comprising of: a) mesenchymal stem cells; b) T regulatory cells; c) type 2 monocytes; d) CD5 positive B cells; e) type 2 NKT cells; f) tolerogenic dendritic cells; g) gamma delta T cells; h) T cells with immune regulatory properties; i) CD34 cells; j) very small embryonic like stem cells and k) Sertoli cells. Methods wherein said mesenchymal stem cell is derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

Methods wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Methods wherein said mesenchymal stem cells do not express substantial levels of HLA-DR, CD117, and CD45. Methods wherein said mesenchymal stem cells are generated from a pluripotent stem cell. Methods wherein said pluripotent stem cell is selected from a group comprising of: a) an embryonic stem cell; b) an inducible pluripotent stem cell; c) a parthenogenic stem cell; and d) a somatic cell nuclear transfer derived stem cell. Methods wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Methods wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging. Methods wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81. Methods wherein said somatic cell nuclear transfer derived stem cells possess a phenotype negative for SSEA-1 and positive for SSEA-3, SSEA-4, TRA-1-60, TRA-1-81, and alkaline phosphatase. Methods wherein said mesenchymal stem cells are differentiated from a pluripotent stem cell source through culture in the presence of an inhibitor of the SMAD-2/3 pathway. Methods wherein said mesenchymal stem cells are differentiated from a pluripotent stem cell source through culture in the presence of an inhibitor nucleic acid targeting the SMAD-2/3 pathway. Methods wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme. Methods wherein said inhibitor of the SMAD-2/3 pathway is a small molecule inhibitor. Methods wherein said small molecule inhibitor is SB-431542.

Methods wherein a selection process is used to enrich for mesenchymal stem cells differentiated from said pluripotent stem cell population. Methods wherein said enrichment method comprises of positively selecting for cells expressing a marker associated with mesenchymal stem cells. Methods wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Methods wherein said immune modulatory cells are autologous, allogeneic or xenogeneic to the recipient. Methods wherein one or more cells are cocultured to generate said conditioned media based on specific need for immune modulation in said recipient. Methods wherein an antigen is administered in combination with said conditioned media. Methods wherein said antigen is selected from a group comprising of: a) a protein; b) a peptide; and c) an altered peptide ligand.

Methods wherein perilymphatic administration is performed by a subcutaneous injection proximal to an area that drains into one or a plurality of lymph nodes. Methods wherein said perilymphatic administration is performed by a intradermal injection proximal to an area that drains into one or a plurality of lymph nodes. Methods wherein said area that drains into one or a plurality of lymph nodes is identified by manual means. Methods wherein said area that drains into one or a plurality of lymph nodes is identified by a visualization means. Methods wherein said visualization means is comprised of lymphangiography. Methods wherein perilymphatic administration is considered intralymphatic administration. Methods wherein said conditioned media generated from cells possessing immune modulatory activity are administered into a superficial inguinal lymph node asceptically. Methods wherein said conditioned media possessing immune modulatory activity are administered in a volume of approximately 0.1 ml. Methods wherein said conditioned media possessing immune modulatory activity are administered slowly under ultrasound guidance. Methods wherein small aspiration of injection site is first performed prior to intra or perilymphatic administration of said cells possessing immune modulatory activity, so as to avoid inadvertent intravascular administration.

Methods wherein said conditioned media are administered into several sites intralymphatically or perilymphatically. Methods wherein said administration is performed according to a frequency of administration required to evoke a desired therapeutic response. Methods wherein said administration is performed on a daily basis. Methods wherein said administration is performed every second day. Methods wherein said administration is performed on a weekly basis. Methods wherein said conditioned media is administered by injection into a dorsal pedal lymphatic channel. Methods wherein said injection into a dorsal pedal lymphatic channel is isolated after Evans blue is infiltrated and lidocaine hydrochloride is administered for local anesthesia according to standard methods used for lymphangiography. Methods wherein said conditioned media is concentrated prior to administration. Methods wherein said concentration of conditioned media is performed by dialysis. Methods wherein said concentration of conditioned media is performed by lyophilization. Methods wherein said concentration of conditioned media is performed by column chromatography. Methods wherein desalting is performed prior to concentration of said conditioned media. Methods wherein desalting is performed subsequent to concentration of said conditioned media.

### DETAILED DESCRIPTION

The disclosed Reference will now be made in detail to exemplary embodiments of the current invention. While the invention will be described in conjunction with these embodiments, it is to be understood that the described embodiments are not intended to limit the invention solely and specifically to only these embodiments that will be mentioned. On the contrary, the invention is intended to cover alternatives, modifications, and equivalents that may be included within the spirit and scope of the invention as defined by the claims.

The present technology encompasses methods for treating of autoimmune conditions and states of immune dysregulation such as rejection of a transplanted cell or organ, or graft versus host disease in a mammal. Aspects of the invention include a method for altering immune responses through the utilization of cells as an immunomodulatory agent. In one embodiment mesenchymal stem cells are generated under Good Manufacturing Conditions (GMP) and administered at a concentration sufficient to elicit an immune modulatory response that is therapeutic to the autoimmune condition of interest via perilymphatic or intralymphatic routes. Within the practice of the current invention is the treatment of autoimmune conditions associated with T cell autoreactivity, particularly conditions associated with type 1 (Th1), type 9 (Th9), or type 17 (Th17) immune responses.

Cell concentrations for administration vary according to specific autoimmune condition, stage of condition, and characteristics of the patient. In one embodiment, a total dose of 0.5 million-300 million mesenchymal stem cells is administered intralymphatically, or perilymphatically to a patient in need of immune modulation, more specifically, to a patient suffering from autoimmunity. Additionally, cell concentrations or types of cells useful for the practice of the invention may be determined by assessment of production of immune modulatory products. One family of immune modulatory products includes the prostanoids. Prostanoids include any of a group of complex fatty acids derived from arachidonic acid, including the prostaglandins, prostanoic acid, and the thromboxanes. Examples of prostanoids of interest include Prostaglandin A, Prostaglandin B, Prostaglandin C, Prostaglandin D, Prostaglandin D2, Prostaglandin E1, Prostaglandin E2, Prostaglandin E2G, Prostaglandin F-alpha, Prostaglandin G, Prostaglandin I, Prostaglandin I2, Prostaglandin J, Prostaglandin K, Thromboxane A2, and Thromboxane B2. In one particular embodiment, the concentration of PGE-2 is utilized as a marker of immune suppressive activity of administered cells, of particular interest, the concentration of PGE-2 produced by mesenchymal stem cells is utilized as a marker of immune suppressive activity of mesenchymal stem cells, said immune suppressive activity is associated with inhibition of autoimmune activity. The frequency of mesenchymal stem cell injection may be performed once, or once a week, or monthly, or yearly. Factors that come into consideration include the stage of autoimmune disease, as well as patient specific factors. Factors of consideration include the amount of T cell autoreactivity that is ongoing as part of the autoimmune process. Specifically T cell autoreactivity may be assessed utilizing CD8 tetramers and flow cytometry, with said tetramers bearing autoantigen. Quantification of autoreactive T cell numbers may be performed by flow cytometry. Activation may be assessed by culture with said autoantigen and assessment of proliferation or cytokine production. Methods are known in the art for assessment of proliferation and autoantigen specific cytokine production such as thymidine incorporation and ELISPOT, respectively. Additional methods of assessing cytokine production include ELISA, Luminex, RT-PCR, Northern Blot and microarrays. Cytokines of interest include ones of specific relevance to autoimmunity including BLC, Eotaxin-1, Eotaxin-2, G-CSF, GM-CSF, I-309, ICAM-1, IFN-gamma, IL-1 alpha, IL-1 beta, IL-1 ra, IL-2, IL-4, IL-5, IL-6, IL-6 sR, IL-7, IL-8, IL-10, IL-11, IL-12 p40, IL-12 p70, IL-13, IL-15, IL-16, IL-17, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, P1GF, SCF, SCF R, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MT, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Angiostatin, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CAl25, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-1ACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK.

One of skill in the art would understand that given the propensity of mesenchymal stem cells to inhibit inflammatory mediators such as TNF-alpha, IFN-gamma, and stimulate anti-inflammatory proteins such as IL-4 and PSG1, in one aspect of the invention, mesenchymal stem cells would be administered at a concentration and frequency to inhibit ongoing inflammation. For example, patients with higher levels of autoreactive T cells producing IFN-gamma would be treated with a higher number of mesenchymal stem cells, and/or at a higher frequency of administration as compared to patients with lower autoreactive T cells. Dosing may also be determined based on clinical characteristics such as stage of the disease.

In one embodiment of the invention, Wharton's jelly mesenchymal stem cells are used as a source of immune modulatory cells. Wharton's jelly cells are derived from umbilical cords that are obtained from healthy mothers that have no history of genetic diseases or cancer, and have been tested negative for hepatitis B/C virus, human immunodeficiency virus, Epstein-Barr virus, cytomegalovirus and syphilis in serum. Manufacturing of Wharton's jelly mesenchymal stem cells is performed in under sterile conditions, for example in a laminar flow hood. During the process of manufacturing, it is ideal for the production to occur in a class 10,000 clean production suite. Each technician properly gowns when entering in the GMP room. Before entry into the clean lab area, the technician obtains a bunny suit in the ante room. After the hood of the bunny suit is placed on, a mouth covering is put on, making sure that all hair is fully covered under the hood and mouth covering. The technician puts on a pair of sterile powder free gloves, and entera the clean lab space with the sample. Environmental monitoring is performed in the Class 10,000 clean room. The umbilical cord is washed with phosphate buffered saline (PBS) twice and then dissected with scissors into pieces approximately one cubic centimeter in volume. The tissue is subsequently plated into a culture dish in low-DMEM medium supplemented with 5-10% platelet rich plasma or fetal calf serum. Cell cultures are maintained in a humidified atmosphere with 5% CO2 at 37° C. After approximately 3 days of culture, the medium is replaced to remove the tissue and non-adherent cells, and the media is changed twice weekly thereafter. Once 80% confluence is reached, the adherent cells (passage 0) were detached with approximately 0.125% trypsin and passaged in the cell culture dish. The Wharton's jelly mesenchymal stem cells are cultured and expanded for 4-6 passages to prepare final cell products. The cellular product is assessed for contamination, including aerobic and anaerobic bacteria, mycoplasma, HBV, HCV, HIV, EBV, CMV, syphilis, and endotoxin testing. To assess purity, cells must possess >90% expression of CD90 and CD105 and <5% CD34, CD45 and HLA-DR. Additionally, cells must have a chromosomal karyotype of UC-MSC was normal.

For production of mesenchymal stem cells, reagent qualification may be necessary. The qualification process begins with the vender of the reagent. The vender is qualified through our standard operating procedure. A corresponding form is completed and approval gained before a vender can be used. The Criteria identified as important in qualifying a supplier include quality of product, services offered, competitive pricing, communication, availability, how complaints are handled and the overall fit to our systems. This list is not all inclusive. Quality Systems reviews each qualification form and will approve based on the criteria stated above. Once the vender is approved, they are added to the Supplies and Services List. Associates ordering supplies including reagents use the list. Only approved venders on the list are used by associates ordering supplies involving reagents. Once the reagent arrives, it is logged on the Supplies Receipt, Inspection and Inventory Log. The form instructs the associate to complete certain information for the incoming reagent. These fields are date received, initials of receiver, name of the item, manufacturer, lot number, expiration date, package passed visual inspection, product passed visual inspection, date available for use and quantity. The COA is examined for reagents and placed in the applicable COA binder under that reagent name. These binders are retained per the record retention procedure. Once this is completed the reagent is released from quarantine and placed in the applicable area. If the reagent needs refrigerated or is to remain frozen, it is placed in the applicable storage environment. FDA or other national regulatory body-approved reagents are used if available. In one embodiment, an excipient used in the cryopreservation of the cells is Dimethyl Sulfoxide (DMSO). Each dose of mesenchymal stem cell may be cryopreserved using 10% DMSO, or 2 mL of DMSO in a total volume of 10 mL of final product. Infusion of this amount of DMSO is well within the safety parameters for a 30 kg child; Pediatric Stem Cell Transplant SOP states that the maximum dose of DMSO is 15 mg/kg/dose. For intralymphatic, or perilymphatic administration, various amounts of cells may be used, as well as numerous lymphatic locations.

In addition to mesenchymal stem cells, the invention may be practiced by administration of Sertoli cells via perilymphatic, or intralymphatic administration. One of skill in the art is directed towards means and methods of isolating Sertoli cells within the scope of the invention, include, patent documents, WO 95/28167, WO 96/28174, WO 98/28030, WO 00/27409, WO 2000/035371, WO 2005/018540, US Pat. App. Pub. 2005/0118145 and U.S. Pat. Nos. 5,725,854, 5,843,340, 5,849,285, 5,948,422, 5,958,404, 6,149,907, 6,303,355, 6,649,160, 6,716,246, 6,783,964, 6,790,441, and 6,958,158.

In some embodiments, the Sertoli cells used for the practice of the invention are adult Sertoli cells. The term “adult”, as used herein, refers to age of a sexually mature male from which the cells are extracted. For this disclosure, sexual maturity is the developmental stage at which a being can reproduce, for example, male rats reach sexual maturity at 3 months, male mice reach sexual maturity at 5-7 weeks and male pigs reach sexual maturity at 6-9 months of age. In illustrative embodiments, the Sertoli cells are porcine cells derived from about 1 to 2 year old boars. Alternatively, the Sertoli cells of the invention may be obtained from any suitable source, for example, cows, horses, dogs, cats, rabbits, primates (human or non-human (e.g., monkeys, chimpanzees)), etc. In other embodiments, Sertoli cells may be derived from a neonatal or fetal animal. Furthermore, Sertoli cells may be generated from stem cells, such as from bone marrow, embryonic stem cells, inducible pluripotent stem cells, or somatic cell nuclear transfer generated stem cells. In some embodiments, the Sertoli cells of the invention have been selected based on expression of immune suppressive molecules, for example Fas ligand. The isolated Sertoli cells may and often do contain other cell types naturally present in the testes, including endothelial cells, Leydig cells, etc. Accordingly, pharmaceutical compositions of the invention may further comprise non-Sertoli cells, including cells that are naturally present in the testes and are, therefore, co-isolated with Sertoli cells. Furthermore, the Sertoli cells of the invention may be primary cells or cell lines derived from such primary cells.

Sertoli cells of the invention may be genetically altered, for example, they may be genetically modified to express, and optionally, secrete one or more immune modulatory factors. Examples of such factors include BLC, Eotaxin-1, Eotaxin-2, G-CSF, GM-CSF, I-309, ICAM-1, IL-1 ra, IL-2, IL-4, IL-5, IL-6 sR, IL-7, IL-10, IL-13, IL-16, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-LACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK. Methods for cell transfection and transformation are known to one of skill in the art. Methods of gene therapy by transfection of genes into Sertoli cells are described, for example, in Dufour et al., Cell Transplant. (2004) 13(1):1-6 and Trivedi et al., Exp. Neurol. (2006) 198, 88-100.

Additionally, pharmaceutical compositions of the invention may comprise non-testicular cells. For example, Sertoli cells may be co-cultured and/or transplanted with another cell type, which benefits from the immunoprotective effect of the Sertoli cells. Specific examples of such other cell types include those that either naturally produce or were modified to produce immune modulatory factors, such as those listed above. In some embodiments Sertoli cells are administered with autoantigens for the purpose of inducing antigen-specific tolerance.

In one specific embodiment, Sertoli cells are isolated from neonatal pigs. Protocols are known in the art for this process. One example involves aseptically excising testicles from a neonatal pig and placing them in a sterile stainless steel pot containing sterile 0.9% saline. The vas deferens and epididymis are trimmed off from the testes, leaving the tunica albuginea intact. The tunica albuginea is then removed and the testes tissue weighed and minced into 1-2 mm fragments. The tissue is then transferred to a 50 ml centrifuge tube with 30-40 ml of HBSS transport media. The contents of the tube is mixed by gently inverting 4 times, then is allowed to sediment by gravity for 5 min. All but 5 ml of media above the pellet is removed and the tissue transferred to a sterile 100 ml Pyrex media bottle with 40 glass beads (2 mm). Digestion is carried out in HBSS (10 mL/g of testicle, without phenol-Red) containing 2.5 mg/ml collagenase and 0.15 mg/m DNase I solution in the shaking water bath at 37.degree. C. set to 200 rpm for 3-5 min. To determine the required length of the digestion, a 10 .mu.l sample aliquot of the digest is mixed 1:1 with trypan blue after 3 min, and every 2 min thereafter. The reaction is stopped when the length of the tubules is 5150 .mu.m. 30-40 ml of HBSS with FBS being added to inactivate the collagenase. The digest is sieved with a 400 .mu.m mesh. The samples are centrifuged at 400.times.g for 4 min at 4.degree. C. The supernatant is then removed and the cell pellet is resuspended in 50 mL of HBSS/FBS. The centrifugation and wash steps are repeated two more times, resulting in a total of 4 washes. Cells are resuspended in complete media (DMEM with 10% bovine serum and 1% penicillin/streptomycin), counted and viability checked with typan blue (typically >95%). 25-30.times.10.sup.6 isolated Sertoli cells are then cultured overnight in T75 culture flasks (Falcon) in 25-30 ml of complete culture media at 37.degree. C. and 5% CO.sub.2 and suitable for utilization for the practice of the invention. In another embodiment, adult porcine Sertoli cells are utilized. Testicles are excised aseptically and the vas deferens and epididymis trimmed, leaving the tunica albuginea intact. Tissues is subsequently transported to the isolation facility on ice in HBSS transport media. Approximately 10 g of tissue is obtained from the testicle. The tissue is minced into 1-2 mm fragments and digestion is performed with 100 mL of filter sterilized (0.2 .mu.m) 2.5 mg/ml collagenase (Type V, Sigma) and 0.15 mg/ml DNase I (Sigma) in HBSS (w/o phenol red, CellGrow). The tissue is transferred to 2 sterile 100 ml Pyrex media bottles each with 40 glass beads (2 mm) and incubated in a shaking water bath at 37.degree. C. set to 200 rpm for 3-15 min. The reaction is stopped when the length of the tubules was .ltoreq.150 .mu.m as determined by microscopic examination. Approximately 30-40 ml of HBSS with FBS is added to inactivate the collagenase and the digest is sieved with a 400 .mu.m mesh. The cells were then transferred into 2.times.50 ml conical tubes and resuspended 4 times with a 10 ml pipette. The total volume is then brought to about 45 ml per tube with the HBSS. The samples are centrifuged at 700 g for 15 min at 4.degree. C. and the pellets are then washed 3 times with 50 mL of HBSS. Cells greater than 3 .mu.m in diameter are counted and viability staining performed on all preparations using typan blue (viability typically >95%). 25.times.10.sup.6 cells (size>3 um) are seeded into 75 cm.sup.2 culture flasks in 25-30 mL of complete media and incubated overnight at 37.degree. C. and 5% CO.sub.2.

In another embodiment, Sertoli cells from humans are used for the practice of the invention. A practitioner of the invention is thought that human Sertoli cells express biochemical markers such as follicle stimulating hormone receptor (FSHr) and GATA4 which may be used for their isolation. Confirmation may be provided by ultrastructural studies that demonstrate the presence of smooth endoplasmic reticulum and perinucleolar spheres as unique features of Sertoli cells. Known properties of Sertoli cells include that conditioned medium from Sertoli cell cultures has the ability to inhibit the proliferation of a human lymphocyte cell line, and that despite proliferation, these cells maintain their immune-privileged ability in culture. The cultured Sertoli cells are known to proliferate in vitro under normal growth conditions (in the absence of any hormone treatment). The rate of proliferation is doubling approximately every 4 days. In addition, the Sertoli cells demonstrate growth inhibition from compaction and cell-cell contact, characteristics of primary cells that have the potential to proliferate. In one embodiment of the invention testes are obtained from cadaveric adult males. The testicular tissue is transferred to 150 mm tissue culture dish and washed with ice cold Hank's Balanced Salt Solution (HBSS) containing 100 U/ml penicillin and 100 .mu.g/ml streptomycin. The dense collagenous connective tissue, the tunica albuginea, is removed using a scissors and the tissue is transferred to a fresh petri dish and rinsed several times with HBSS and minced into tiny pieces of approximately 5-10 mm. The minced tissue is transferred to 1,000-ml Erlenmeyer flask, washed three times with HBSS discarding the media after each wash, and then covered with HBSS and transferred to a 37.degree. C. water bath and shaken at 325 rpm for 15 min. The tissue is allowed to settle, the supernatant is discarded, and 50-ml of HBSS containing 0.25% trypsin (Sigma, St. Louis, Mo.), 0.1% collagenase Type IV (Sigma) and 2.4 .mu.U dispase/ml (Roche, Indianapolis, Ind.) is added. The flask is shaken at 325 rpm at 37.degree. C. for 20 min. Then the solution is strained through a coarse wire mesh, the flow-through stored on ice, and the undigested tissue pieces are again placed in HBSS containing the same enzyme mixture, and shaken at 325 rpm at 37.degree. C. for 15 min. These steps are repeated until most of the tissue is digested. Finally, 0.034% of soybean trypsin inhibitor (Sigma) is added. The solution is passed through a syringe with an 18-gauge needle, and then centrifuged at 800.times.g for 5 min. The supernatant is discarded and the cell pellet resuspended in tissue culture medium (Dulbeccos Modified Eagle medium (DMEM):F-12 Hams medium, 50:50, containing 100 U/ml penicillin and 100 .mu.g/ml streptomycin, 5% fetal bovine serum) and plated in a T-225 flask. Cell viability is determined by exclusion of trypan blue dye. The cells are propagated in the same medium containing 5% fetal bovine serum, and incubated at 37.degree. C. in a 5% CO.sub.2 incubator. The cells reached confluence 3-5 weeks after first observing cells adhering to the flask at which point they were passaged. Some of the cells are frozen in cell preservation medium and stored under liquid nitrogen. Light microscopy is performed using an inverted Olympus microscope and observation of the culture flask containing the cell pellet revealed only cell debris and many dead cells over the first few days to weeks. No cells adhering to the surface of the flask are observed. At 2-20 days post-isolation, a few thin, long cells are observed adhering to the bottom of the culture flasks. At first all that could be seen is few groups that each contained only a few (2-10) cells. But within a few days of the first observation, the cells begin to flatten out and their bodies became polygonal with extensive branching cytoplasmic structures that are characteristics indicative of Sertoli cells. The groups of cells also began to multiply locally, but in addition a few cells or groups of cells were observed at locations well-separated (5-10 cm) from any other cells. The first observation of adherent cells appears at approximately 3 weeks after the isolation procedure is performed. Characteristic features of Sertoli cells include a large irregularly-shaped nucleus, extensive and branching cytoplasmic structures, prominent nucleoli, perinucleolar spheres, lipid droplets, and abundant smooth and rough endoplasmic reticulum. The oval to pyramidal shape of the nucleus and the extensive and branching cytoplasmic structure of the cells can be observed in bright-field photomicrographs.

Various populations of mesenchymal stem cells may be used for the practice of the invention, in addition to bone marrow, adipose, or umbilical cord derived mesenchymal stem cells, amniotic membrane mesenchymal stem cells may be utilized as immune modulatory cells. In one specific embodiment, 8_{—}8 cm2 sections of amniotic membrane are obtained. They were washed with 1.0M phosphate-buffered saline (PBS; pH 7.2) containing 300 IU/ml penicillin and 300 mg/ml streptomycin (Gibco, Grand Island, N.Y., USA), and are immediately immersed in Dulbecco's modified Eagle's medium (DMEM)-high glucose (Gibco), supplemented with 10% fetal bovine serum (FBS; Gibco), 300 IIU/ml penicillin and 300 mg/ml streptomycin. All samples are processed within 12-15 h after collection. The amniotic membranes are treated with 0.1% collagenase I (Sigma-Aldrich, St Louis, Mo., USA) in 1.0M PBS (pH 7.2) and are incubated at 37_C for 20 min. Each amniotic membrane is washed three times with low-glucose DMEM (Gibco), and the detached cells are harvested after a gentle massage of the amniotic membrane. The cells are centrifuged at 300 g for 10 min at 37_C, and subsequently resuspended in RPMI 1640 medium with 10% FBS, then grown in 25 cm2 flasks at a density of 1_{—}10⁶ cells/ml. After 24 h incubation, nonadherent cells are removed. The culture medium is replaced every 3 days. Adherent cells are cultured until they reached 80-90% confluence. Cells are subsequently selected based on quality control procedures including purity (eg >90% CD90 and CD105 positive), sterility (eg lack of endotoxin and mycoplasma/bacterial contamination) and potency (eg ability to immune modulate in vitro by suppressing production of inflammatory cytokines such as IFN-gamma). Cells may subsequently be utilized for perilymphatic or intralymphatic administration. The present application contemplates the collection and delivery of a naturally occurring population of MSC derived from intra alia, placental/umbilical cord, bone marrow, skin, or tooth pulp tissue. In accordance with the invention, the MSCs are generally an adherent cell population expressing markers CD90 and CD105 (>90%) and lacking expression of CD34 and CD45 and MHC class II (<5%) as detected by flow cytometry, although other markers described in the specification may be utilized.

In the case of placental tissue, which represents an almost unlimited supply of MSC, placenta are collected from delivery procedures, the tissue may be placed in sterile containers with phosphate buffered saline (“PBS”), penicillin/streptomycin and amphotericin B during collection. This may be performed when collecting testicular or ovarian tissue as well. Specifically, harvested tissue is first surface sterilized by multiple washes with sterile PBS, followed by immersion in 1% povidoneiodine (“PVP-1”) for approximately 2 minutes, immersion in 0.1% sodium thiosulfate in PBS for approximately 1 minute, and another wash in sterile PBS. Next the tissue is dissected into 5 g pieces for digestion. Enzymatic digestion is performed using a mixture of collagenase type I and type II along with thermolysin as a neutral protease. The digestion occurs in a 50 cc sterile chamber for 20-45 minutes until the tissue is disaggregated and the suspending solution is turbid with cells. Next the solution is extracted leaving behind the matrix, and cold (4.degree. C.) balanced salt solution with fetal bovine serum at 5% concentration is added to quench the enzymes. This resulting suspension is centrifuged at 600.times.g, supernatant is aspirated and MESENCULT® complete medium (basal medium containing MSC stimulatory supplements available from StemCell Technologies, Vancouver, British Columbia) is added to a final volume of approximately 1.5 times the digestion volume to neutralize the digestion enzymes. This mixture is centrifuged at 500 g for 5 minutes, and the supernatant aspirated. The cell pellet is be re-suspended in fresh 10 MESENCULT® complete medium plus 0.25 mg/mL amphotericin B, 100 IU/mL penicillin-G, and 100 mg/mL streptomycin (JR Scientific, Woodland, Calif.).

Cells are then plated at an initial concentration of approximately one starting 5 g tissue digest per 225 cm2 flask. Culture flasks are monitored daily and any contaminated flasks removed immediately and recorded. Non-contaminated flasks are monitored for cell growth, with medium changes taking place three times per week. After 14 days of growth, MSC are detached using 0.25% trypsin/1 mM EDTA (available from Invitrogen, Carlsbad, Calif.). Cell counts and viability were assessed using flow cytometry techniques and cells are banked by controlled rate freezing in sealed vials. For the preparation of bone marrow MSC, bone marrow is collected and placed within a “washing tube”. Before the collection procedure a “washing tube” is prepared in the class 100 Biological Safety Cabinet in a Class 10,000 GMP Clean Room. To prepare the washing tube, 0.2 mL amphotericin B (Sigma-Aldrich, St Louis, Mo.), 0.2 mL penicillin/streptomycin (Sigma 50 ug/nl) and 0.1 mL EDTANa2 (Sigma) is added to a 50 mL conical tube (Nunc) containing 40 mL of GMP-grade phosphate buffered saline (PBS). Specifically, the washing tube containing the collected bone marrow is topped up to 50 mL with PBS in a class 100 Biological Safety Cabinet and cells are washed by centrifugation at 500 g for 10 minutes at room temperature, which produced a cell pellet at the bottom of the conical tube. Under sterile conditions supernatant is decanted and the cell pellet is gently dissociated by tapping until the pellet appeared liquid. The pellet is re-suspended in 25 mL of PBS and gently mixed so as to produce a uniform mixture of cells in 30 PBS. In order to purify mononuclear cells, 15 mL of Ficoll-Paque (Fisher Scientific, Portsmouth N.H.) density gradient was added underneath the cell-PBS mixture using a 15 mL pipette. The mixture is subsequently centrifuged for 20 minutes at 900 g. Thereafter, the buffy coat is collected and placed into another 50 mL conical tube together with 40 mL of PBS. Cells are then centrifuged at 400 g for 10 minutes, after which the supernatant is decanted and the cell pellet re-suspended in 40 mL of PBS and centrifuged again for 10 minutes at 400 g. The cell pellet is subsequently re-suspended in 5 mL complete DMEM-low glucose media (GibcoBRL, Grand Island, N.Y.) supplemented with approximately 20% Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot is used for all experiments. Additionally, the media is supplemented with 1% penicillin/streptomycin, 1% amphotericin B, and 1% glutamine. The re-suspended cells are mononuclear cells substantially free of erythrocytes and polymorphonuclear leukocytes as assessed by visual morphology microscopically. Viability of the cells was assessed with trypan blue. Only samples with >90% viability were selected for cryopreservation in sealed vials. For preparation of MSC from teeth, said teeth are extracted under sterile conditions and placed into sterile chilled vials containing 20 mL of phosphate buffered saline with penicillin/streptomycin and amphotericin B (Sigma-Aldrich, St. Louis, Mo.). Teeth were thereafter externally sterilized and processed first 20 by washing several times in sterile PBS, followed by immersion in 1% povidoneiodine (PVP-1) for 2 minutes, immersion in 0.1% sodium thiosulfate in PBS for 1 minute, followed by another wash in sterile PBS. The roots of cleaned teeth is separated from the crown using pliers and forceps to reveal the dental pulp, and the pulp is placed into an enzymatic bath consisting of type I and type II collagenase (Vitacyte, Indianapolis, USA) with thermolysin as the neutral protease. Pulp tissue is allowed to incubate at 37.degree. C. for 20-40 min to digest the tissue and liberate the cells. Once digestion is complete, MESENCULT® complete medium is added to a final volume of 1.5.times. the digestion volume to neutralize the digestion enzymes. This mixture is centrifuged at 500 g for 5 min, and the supernatant aspirated. The cell pellet sare resuspended in fresh MESENCULT® complete medium plus 0.25 mg/mL amphotericin B, 100 30 IU/mL penicillin-G, and 100 mg/mL streptomycin (JR Scientific, Woodland, Calif.). Cells are plated at an initial concentration of one tooth digest per 25 cm.sup.2 flask. Culture flasks are monitored daily and any contaminated flasks removed immediately and recorded. Non-contaminated flasks were monitored for cell growth, with medium changes taking place three times per week. After 14 days of growth, MSC are detached using 0.25% trypsin/1 mM EDTA (Invitrogen, Carlsbad, Calif.), cell counts and viability were assessed using a standard trypan blue dye exclusion assay (Sigma) and hemacytometer, and bAU3 the DPSC divided equally between two 75 cm.sup.2 flasks. After the first passage, DPSC cultures were harvested once they reach 7080% confluence. These cells are then cryopreserved in sealed vials. MSCs from the skin, including epidermal, dermal, and subcutaneous tissue of healthy adult patients undergoing cosmetic plastic surgery are isolated by collagenase digestion procedure. Once received, the tissue is cleaned of any unwanted adipose tissue and hair The tissue is then sterilized using 1.times. PVP-iodine solution and 1.times. sodium thiosulfate followed by washing twice in sterile PBS. The dermis is then minced into 1 mm.sup.3 pieces following collagenase enzymatic digestion for 30-40 minutes at 37.degree. C. Afterwards, tissue pieces were dissociated by pipetting into 5 mL pipette and centrifuged at 300 g for 5 min The pellet was suspended in cell growth media Dulbecco's Modified Eagle Medium: Nutrient Mixture F-12 (“DMEM/F12”) (available from Invitrogen, Carlsbad, Calif.) (1:1) containing amphoterecin, penicillin and streptomycin supplemented with 10% fetal bovine serum. Cell suspensions were transferred into T-tissue culture flask and grown until 80-90% confluence. The cells were placed in a T-75 flask before being used for flow analysis and differentiation. Another embodiment of the invention is the use of MSCs from the umbilical cord during harvested during delivery. Once received, the tissue i washed two to three times in sterile PBS and then divided into pieces of approximately 5 grams each. Thereafter, the tissue is decontaminated, and each 5 gram aliquot of tissue is placed in a sterile 100 mm tissue culture dish, and covered with a lid to prevent drying. The tissue was dissociated via enzymatic digestion in 50 cc tubes, and is minced into fragments less than 1 mm.sup.3 using a sterile scalpel. Then, the chopped tissue is placed in an enzyme bath, and the tube is capped and transferred to an incubator. The tubes were swirled for fifteen seconds every ten minutes for forty minutes. Thereafter, the digesting enzyme was diluted by adding 45 mL of cold DME/F12 complete media (FBS, Pen/Strep and Amphotericin B), with the tubes being capped and inverted to mix the contents. Next, the tubes were centrifuged at 400.times.g for fifteen minutes on low break. The top media is aspirated using a 25 mL pipette by leaving approximately 5 mL at the bottom of the tube, with special care being taken to aspirate the entire medium in the tube. The bottom 5 mL medium (containing tissue fragments and cells including MSCs) was resuspended in fresh 20 mL DME-F12 complete medium mixed well and placed into a t-75 flask, and transferred to an incubator. The tissue is washed off during the first media 10 change after 48 hours post-digestion, and the media was changed three times per week. Cells are grown to 70%-80% confluence and then either passaged, frozen down as passage zero cells, or differentiated. Cells were not allowed to reach confluence or to remain at confluence for extended periods of time.

Cell expansion for cells originating from any of the abovementioned tissues above takes place in clean room facilities purpose built for cell therapy manufacture and meeting GMP clean room classification. In a sterile class II biologic safety cabinet located in a class 10,000 clean production suite, cells were thawed under controlled conditions and washed in a 15 mL conical tube with 10 ML of complete DMEM-low glucose media (cDMEM) (GibcoBRL, Grand Island, N.Y.) supplemented with 20% Fetal Bovine Serum (Atlas) from dairy cattle confirmed to have no BSE % Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot was used for all experiments. Cells are subsequently placed in a T-225 flask containing 45 mL of cDMEM and cultured for 24 hours at 37.degree. C. at 5% CO2 in a fully humidified atmosphere. This allowed the MSC to adhere. Non-adherent cells were washed off using cDMEM by gentle rinsing of the flask. This resulted in approximately 6 million cells per initiating T-225 flask. The cells of the first flask were then split into 4 flasks. Cells were grown for 4 days after which approximately 6 million cells per flask were present (24 million cells total). This scheme was repeated but cells were not expanded beyond 10 passages, and were then banked in 6 million cell aliquots in sealed vials for delivery. All processes in the generation, expansion, and product production were performed under conditions and testing that was compliant with current Good Manufacturing Processes and appropriate controls, as well as Guidances issued by the FDA in 1998 Guidance for Industry: Guidance for Human Somatic Cell Therapy and Gene Therapy; the 2008 Guidance for FDA Reviewers and Sponsors Content and Review of Chemistry, Manufacturing, and Control (CMC) Information for Human Somatic Cell Therapy Investigational New Drug Applications (INDs); and the 1993 FDA points-to-consider document for master cell banks were all followed for the generation of the cell products described. Donor cells are collected in sterile conditions, shipped to a contract manufacturing facility, assessed for lack of contamination and expanded. The expanded cells are stored in cryovials of approximately 6 million cells/vial, with approximately 100 vials per donor. At each step of the expansion quality control procedures were in place to ensure lack of contamination or abnormal cell growth.

Without departing from the spirit of the invention, mesenchymal stem cells may be optimized to possess heightened immune modulatory properties. In one embodiment this may be performed by exposure of mesenchymal stem cells to hypoxic conditions, specifically hypoxic conditions can comprise an oxygen level of lower than 10%. In some embodiments, hypoxic conditions comprise up to about 7% oxygen. For example, hypoxic conditions can comprise up to about 7%, up to about 6%, up to about 5%, up to about 4%, up to about 3%, up to about 2%, or up to about 1% oxygen. As another example, hypoxic conditions can comprise up to 7%, up to 6%, up to 5%, up to 4%, up to 3%, up to 2%, or up to 1% oxygen. In some embodiments, hypoxic conditions comprise about 1% oxygen up to about 7% oxygen. For example, hypoxic conditions can comprise about 1% oxygen up to about 7% oxygen; about 2% oxygen up to about 7% oxygen; about 3% oxygen up to about 7% oxygen; about 4% oxygen up to about 7% oxygen; about 5% oxygen up to about 7% oxygen; or about 6% oxygen up to about 7% oxygen. As another example, hypoxic conditions can comprise 1% oxygen up to 7% oxygen; 2% oxygen up to 7% oxygen; 3% oxygen up to 7% oxygen; 4% oxygen up to 7% oxygen; 5% oxygen up to 7% oxygen; or 6% oxygen up to 7% oxygen. As another example, hypoxic conditions can comprise about 1% oxygen up to about 7% oxygen; about 1% oxygen up to about 6% oxygen; about 1% oxygen up to about 5% oxygen; about 1% oxygen up to about 4% oxygen; about 1% oxygen up to about 3% oxygen; or about 1% oxygen up to about 2% oxygen. As another example, hypoxic conditions can comprise 1% oxygen up to 7% oxygen; 1% oxygen up to 6% oxygen; 1% oxygen up to 5% oxygen; 1% oxygen up to 4% oxygen; 1% oxygen up to 3% oxygen; or 1% oxygen up to 2% oxygen. As another example, hypoxic conditions can comprise about 1% oxygen up to about 7% oxygen; about 2% oxygen up to about 6% oxygen; or about 3% oxygen up to about 5% oxygen. As another example, hypoxic conditions can comprise 1% oxygen up to 7% oxygen; 2% oxygen up to 6% oxygen; or 3% oxygen up to 5% oxygen. In some embodiments, hypoxic conditions can comprise no more than about 2% oxygen. For example, hypoxic conditions can comprise no more than 2% oxygen.

Enhancement of immune modulatory activity of mesenchymal stem cells may be performed by altering the oxidative stress levels of the patient before, and/or during, and/or after administration of the cells. In one embodiment the patient is treated using mesenchymal stem cells administered intralymphatically or perilymphatically in combination with enhancing the anti-oxidant status of the patient. Enhancement of antioxidant status may be performed through administration of an antioxidant, or combination of antioxidants, said antioxidant may be selected from a group comprising of: ascorbic acid and derivatives thereof, alpha tocopherol and derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, intravenous ascorbic acid, polyphenols, pycnogenol, retinoic acid, ACE Inhibitory Dipeptide Met-Tyr, recombinant superoxide dismutase, xenogenic superoxide dismutase, and superoxide dismutase.

In some aspects of the invention, a chemoattractant agent or combination of agents are administered either proximally, or directly to the organ being affected by autoimmunity with the purpose of proximally concentrating mesenchymal stem cells to area of inflammation/autoimmunity. Said chemoattractant may be administered in the form of a depot, said depot capable of substantially localizing said chemoattractant is may be selected from a group comprising of: fibrin glue, polymers of polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolifins, and polyvinyl alcohol. Furthermore, said chemoattractants useful for the practice of the current invention may be is selected from a group comprising: SDF-1, VEGF, RANTES, ENA-78, platelet derived factors, various isoforms thereof and small molecule agonists of VEGFR-1, VEGFR2, and CXCR4. In another aspect of the invention, the chemoattractant is administered into the area in need, through transfection of a single or plurality of nucleotide(s) encoding said chemoattractant factor. In some embodiments, a perilymphatic or intralymphatic administration of a chemoattractant factor is administered in order to augment retention of mesenchymal stem cells in the lymphatic area.

In another embodiment, mesenchymal stem cells may be optimized for enhanced trafficking and/or immune modulatory activity by genetic modification. Mesenchymal stem cells that expresses or up-regulates expression of a polypeptide, such as, for example, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors .alpha.sub.1.beta.sub.1 and .alpha.sub.2.beta.sub.1, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor .alpha.5.beta.1, Factor X, HB-EGF, HBNF, HGF, HUAF, heart derived inhibitor of vascular cell proliferation, Ill, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-.beta., PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-.beta., and TGF-.beta. receptors, TIMPs, TNF-alpha, TNF-beta, transferrin, thrombospondin, urokinase, VEGF-A, VEGF-B, VEGF-C, VEGF-D, VEGF-E, VEGF, VEGF.sub.164, VEGI, EG-VEGF, VEGF receptors, PF4, 16 kDa fragment of prolactin, prostaglandins E1 and E2, steroids, heparin, 1-butyryl glycerol (monobutyrin), and/or nicotinic amide. Additionally, mesenchymal stem cells may be transfected with a nucleic acid sequence that induces RNA interference to silence genes associated with pathological immunity such as ABCF1, BCL6, C3, C4A, CEBPB, CRP, ICEBERG, IL1R1, IL1RN, IL8RB, LTB4R, TOLLIP, IFNA2, IL10RA, IL10RB, IL13, IL13RA1, IL5RA, IL9, IL9R, CD40LG (TNFSF5), IFNA2, IL17C, IL1A, 1L1B, 1L1F10, IL1F5, IL1F6, IL1F7, IL1F8, IL1F9, IL22, IL5, IL-6, IL8, IL9, IL-18, IL-33, LTA, LTB, MIF, SCYE1, SPP1, TNF, CCL13 (mcp-4), CCR1, CCR2, CCR3, CCR4, CCR5, CCR6, CCR7, CCR8, CCR9, CX3CR1, IL8RA, XCR1 (CCXCR1), C5, CCL1 (1-309), CCL11 (eotaxin), CCL13 (mcp-4), CCL15 (MIP-1d), CCL16 (HCC-4), CCL17 (TARC), CCL18 (PARC), CCL19, CCL2 (mcp-1), CCL20 (MIP-3a), CCL21 (MIP-2), CCL23 (MPIF-1), CCL24 (MPIF-2/eotaxin-2), CCL25 (TECK), CCL26, CCL3 (MIP-1a), CCL4 (MIP-1b), CCL5 (RANTES), CCL7 (mcp-3), CCL8 (mcp-2), CXCL1, CXCL10 (IP-10), CXCL11 (1-TAC/IP-9), CXCL12 (SDF1), CXCL13, CXCL14, CXCL2, CXCL3, CXCL5 (ENA-78/LIX), CXCL6 (GCP-2), CXCL9, IL13, and IL8. In some embodiments, mesenchymal stem cells are endowed with augmented antiapoptotic activity by transfection nucleic acids that induce gene silencing to suppress expression of genes associated with induction of apoptosis, such genes include CASP1 (ICE), CASP10 (MCH4), CASP14, CASP2, CASP3, CASP4, CASP5, CASP6, CASP7, CASP8, CASP9, CFLAR(CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, and TRAF4. In another embodiment, mesenchymal stem cells are modulated, either by transfection or other means to enhance expression of anti-apoptotic proteins, such proteins include obestatin, XIAP, survivin, BCL-2, BCL-XL, GATA-4, IGF-1, EGF, heme-oxygenase-1, NF-kB, akt, pi3-k, and epha-2.

The use of mesenchymal stem cells for stimulation of the tolerogenic process may also be performed as an adjuvant to other processes, methodologies, or agents that promote immunological tolerance. Within the definition of immunological tolerance includes suppression of an ongoing autoimmune response, promotion of T regulatory cells, B regulatory cells, tolerogenic dendritic cells, NKT2 cells and type 2 macrophages.

One particular embodiment of the invention is the utilization of mesenchymal stem cells as a means of modulating dendritic cell (DC) function in vitro or in vivo. The role of the DC in vivo may be conceptualized in a very general sense as a dual purpose cell: In conditions of homeostasis, DC reside in an immature state and promote tolerance, in contrast, when DC are exposed to injury/damage signals they mature and induce T cell activation. This general paradigm can be observed in the four conditions of tolerogenesis that will be discussed in the specification, particularly pregnancy, cancer, ACAID, and oral tolerance. One of skill in the art will utilize these conditions of natural tolerogenesis to guide the use of mesenchymal stem cells are promoters of the tolerogenic process. The direct use of mesenchymal stem cells as a “reprogrammer” of the immune system via intralymphatic or perilymphatic administration has not been previously contemplated, due to the general thought in the art that this cell population is primarily of a regenerative nature. In pregnancy circulating factors such as TGF-b family members [58] and hCG [59], have been reported to inhibit DC maturation and function [60, 61]. DC with tolerogenic properties are found at the maternal-fetal interface and express high concentrations of the immune suppressive enzyme indolamine 2,3 deoxygenase (IDO). Through local tryptophan depletion, as well as production of immune suppressive metabolites, cells expressing IDO have been demonstrated to induce T cell apoptosis, and more recently to elicit generation of T regulatory (Treg) cells [62, 63]. The critical role of this enzyme in pregnancy can be seen in studies where IDO inhibition results in immunologically mediated spontaneous abortion [64]. Accordingly it is within the scope of the current invention to manipulate in vivo conditions using mesenchymal stem cells so as to generate a tolerance promoting environment similar to that which occurs in conditions of natural tolerogenesis. Particularly, in one embodiment, mesenchymal stem cells are administered together with a physiological concentration of hCG to elicit tolerogenesis. Administration of the mesenchymal stem cells, and/or of the hCG may be intravenous, intralymphatic, or perilymphatic. In another embodiment, mesenchymal stem cells are administered together with TGF-beta to elicit tolerogeneisis. In another embodiment mesenchymal stem cells are administered together with IDO gene therapy to promote tolerogenesis Inhibition of DC maturation and/or reprogramming by the tumor microenvironment has been well documented in numerous clinical system and animal experiments. DC isolated from tumor draining lymph nodes in melanoma [65, 66], ovarian [67], breast [68], and lung cancer [69] have been characterized as having an immature/plasmacytoid phenotype, suppressed T cell activating ability and possess elevated levels of IDO. Manipulation of DC by silencing the gene IDO using siRNA has been demonstrated to evoke productive T cell immunity towards melanoma [70]. Secretion of VEGF by tumor cells is one of several proposed mechanisms for increased immature DC in tumor patients [71]. Administration of the anti-VEGFR antibody bevacizumab in patients with a variety of tumors was demonstrated to increase DC maturation and restore T cell activating activity [72]. Accordingly, within the context of the current invention, mesenchymal stem cells may be administered together with concentrations of VEGF found in the tumor to be tolerogenic.

In some embodiments, the invention discloses means of augmenting natural or induced tolerogenic processes. In the situation of oral tolerance, a population of T cell suppressive CD11c+,CD11b+ DCs and CD11c+,CD8alpha+ DCs has been reported in the Peyer's patches [73]. These cells have been described to express high levels of IDO and possess ability to activate Treg cells [74]. In on embodiment of the invention mesenchymal stem cells are administered proximal or directly into the Peyer's Patches to induce immune regulation. Alternatively, immune modulatory cells may be administered intralymphatically, or perilymphatically, into a mammal having ingested an antigen or plurality of antigents. It is known that administration of flt-3L, which expands DC systemically has been demonstrated to augment effects of oral tolerance induction [75]. Accordingly, in one aspect of the invention, mesenchymal stem cells, or other immune modulatory cells, are administered intralymphatically, or perilymphatically, together with flt-3L as a means of stimulating tolerogenesis. A more recent report described IL-10/IL-27 expressing CD11b-DC as inducers of oral tolerance in a transgenic system. The relationship between these cells and IDO expressing DC remains to be elucidated [76]. Accordingly it is within the scope of the current invention to utilize intralymphatic and perilymphatic mesenchymal stem cell administration to augment oral tolerance induction.

Unique antigen presenting cells bearing the macrophage marker F4/80 reside in the anterior chamber of the eye, whose migration in the spleen and activation of regulatory cells of the NKT lineage is essential for ACAID to occur [77]. The importance of this antigen presenting cell in ACAID can be seen from studies in which similar concentrations of TGF-b as those found in the anterior chamber are added exogenously to naïve monocytes. The resulting cell population, which phenotypically resembles ocular macrophages have the potential to induce immune modulation in vivo through induction of Treg cells [78]. Thus it appears that the process of tolerogenesis is associated with a critical function of the DC/antigen presenting cell. Given this knowledge artificial manipulation of DC for induction of tolerance has been performed in several settings. For example, tolerogenic modifications of DC included exposure of the DC to small molecule immune suppressants [79-81], gene transfection with tolerogenic genes [82, 83] and gene silencing of immune activatory genes [84-87]. These references are provided so that one of skill in the art can derive concentrations and reagents useful for generating tolerogenic or immune modulatory DC which may be utilized together with intralymphatic or perilymphatic administration of mesenchymal stem cells for the purposes of tolerogenesis.

It has been previously reported [88], immature dendritic cells possessing tolerogenic function, termed Tol-DC, have the ability to induce generation of T regulatory (Treg) cells. One embodiment of the current invention involves utilization of mesenchymal stem cells administered intralymphatically and perilymphatically to induce generation of Tol-DC, which in turn stimulate Treg formation, or alternatively to directly induce Treg formation.

A background on Treg cells will be provided to one of skill of the art a starting point for practice of the invention in light of stimulation of Treg for inhibition of autoimmunity. The concept of T cells suppressing other T cells as a mechanism of tolerance was accepted for decades. Initial studies in the 1970s focused on “T suppressor” cells, which were CD8 positive cells with the ability to restrain autoimmunity, support transplant tolerance, and were elevated in cancer. The existence of these cells came into doubt when molecular studies demonstrated fundamental proteins ascribed to these cells could not be found [89]. In the 1990s the focus started to shift to cells expressing the CD4+, CD25+ phenotype. The group of Hall et al were the first to describe a cell population with this phenotype capable of transferring tolerance in a rat model of transplantation [90, 91]. Subsequently, Sakaguchi's group, which are commonly given credit for identification of the Treg cell, confirmed the importance of the CD4+ CD25+ phenotype based on experiments demonstrating neonatal thymectomy causes loss of Treg, which results in systemic autoimmunity, which is prevented by transfer of the cell population [92]. Since those early days, the field of Treg has blossomed, with numerous molecular details of their function having been elucidated. Interestingly, observations made with the ill-defined T suppressor cells in the early 1980s, such as ability to suppress antigen presenting cell function [93], are now being rediscovered with Treg cells [88]. Accordingly, in one embodiment of the invention, immune modulatory cells, such as amniotic membrane derived cells, umbilical cord mesenchymal stem cells, or Sertoli cells are administered intralymphatically or perilympatically to induce Tol-DC in vivo, in other embodiments the cells are used in vitro to generate Tol-DC. In one specific embodiment, peripheral blood mononuclear cells (PBMCs) are isolated from leukapheresis products using a commercially available apheresis system. The leukapheresis product is loaded via the inlet pump into the constantly rotating elutriation chamber. The automation mode produces approximately five elutriation fractions, each specified by centrifuge speed, loading or elutriation buffer flow rate, and process volume. The final monocyte-rich fraction (Fraction 5) is then collected from the chamber into the final collection bag when the centrifuge is stopped. All procedures are conducted according to the manufacturer's recommendations, except that Hanks' buffered salt solution (HBSS) is used an elutriation buffer. Enriched monocytes are then cultured on T75 culture flasks in X-VIVO 15 media, 20 μg/mL gentamicin, 2 mM glutamine, 5% heat-inactivated AB human plasma, 250 ng/mL recombinant human (rHu) GM-CSF, and 20 ng/mL rHu IL-4. Cultures are fed every other day by removing half of the supernatant and adding fresh medium with full doses of cytokines. On day 6, immature DC (iDC) were generated and harvested. Immature DC are subsequently cultured together with mesenchymal stem cells at a 1:1 ratio for 24-96 hours, more optimally for approximately 48 hours. Subsequent to cultures, the DC and mesenchymal stem cells mixture are administered intravenously, or more preferably, intralymphatically or perilymphatically. In some embodiments the monocyte-immature DC is pulsed with autoantigen. The ability to pulse the DC with autoantigen allows for induction of antigen-specific tolerance. The presence of the mesenchymal stem cells in the co-culture ensures the DC remain in an immature state.

Within the context of the invention, it is known that in the skin, initial lymphatic vessels are localized beneath the epidermis and serve as a conduit for cellular migration from the skin to the draining lymph nodes. Migration of cells to and through the lymphatic vessels after intradermal administration is primarily guided by chemokine gradients of CCL19/CCL21 secreted by lymphatic endothelial cells under a physiologic condition [94]. Mesenchymal stem cells are known to express the CCL19/CCL21 receptor CCR7 [95]. Thus in one embodiment, immune modulatory cells are pretreated prior to administration into a recipient, in a manner to augment migratory ability towards CCL19/CCL21 gradients. Such treatments include means of augmenting expression of the protein CCR7. Specific means include exposure to hypoxia, which is described in the art to augment migratory activity [96].

In addition to chemokines, it is known that the intrinsic cellular migratory ability and dermal tissue microenvironment are also pivotal in the control of migration from the skin to the draining lymph nodes. In the case of dendritic cells, it has been shown that, a lack of MHC II-associated invariant chain (li or CD74) is associated with increased dendritic cell motility in vitro and in vivo due to decreased adhesion of the dendritic cell to the matrix of the skin connective tissue [97, 98]. As part of migration from skin to draining lymph nodes, cells require expression of matrix metalloproteases (MMPS) to cross extracellular matrix. This is apparent in the case of dendritic cells in that lack of MMP2 and MMP9 has been demonstrated to reduce migration into draining lymph nodes after intradermal administration [99, 100]. Accordingly, it is within the scope of the current invention to provide means of augmenting MMP expression within immune modulatory cells utilized for the practice of the invention, said means of augmenting MMP expression include transfection with relevant MMPs, such as MMP2, MMP3, MMP7, MMP9, and MMP10, as well as culture in conditions stimulating upregulation of said MMPs.

The interstitial space of the dermis provides for an open one-way communication with the lymphatic system. A constant interstitial flux attracts serum components filtrated from the capillary bed toward the initial lymphatic vessels that possess flap valves to allow entry but prevent exit of solutes and small particles. Patrolling immune cells and stem cells follow the same principal route. Whereas it is known that fluid flux is driven by the periodic contractions of the lymphatic suction pump, cells rely on their own mechanisms and chemoattractant gradients for crawling through the interstitium toward and into the initial lymphatic vessel.

In one embodiment cells are injected proximal to lymphatics that drain into lymph nodes. Perilymphatic administration is described as administration into the lymphatics that drain into the lymph nodes. In one embodiment, administration is performed unilaterally into lymphatics that drain into lymph nodes on one side of the body. In other embodiments, administration is performed bilaterally, that is, on both sides of the body. Numerous techniques are known in the art for administration into lymph nodes or into lymphatics that drain into lymph nodes. In one embodiment, administration of cells, or conditioned media from cells, or the combination is administered utilizing a 26 gauge needle at a depth of approximately 15 mm in the anterior margin of the sternocleidomastoid muscle at 15 mm from its insertion on the mastoid.

In another embodiment, the superficial inguinal lymph node is aseptically and slowly injected under ultrasound guidance with a solution of approximately 0.1 ml of cells. A small aspiration is first made before injection in order not to inadvertently induce intravascular administration. In another embodiment, cells are administered into the dorsal pedal lymphatic channel.

In one embodiment of the invention, conditioned media from immune modulatory cells is utilized in place of the cells. Conditioned media is generated by contacting viable immune modulatory cells described in the application, for example mesenchymal stem cells. In one embodiment, the invention provides a means of creating a medicament to be administered intralymphatically or perilymphatically useful for the treatment of inflammatory, autoimmune, and degenerative conditions through culturing Wharton Jelly mesenchymal cells in a serum free media. Many types of media may be used and chosen by one of skill in the art. In one embodiment a media is selected from a group comprising of alpha MEM, DMEM, RPMI, Opti-MEM, IMEM, and AIM-V. Cells may be cultured in a variety of media for expansion that contain fetal calf serum, or other growth factors, however, for collection of therapeutic supernatant, in a preferred embodiment, the cells are transferred to a media substantially lacking serum. It is well known in the art that preparation of the supernatant before administration may be performed by various means, for example, said supernatant may be filter sterilized, or in some conditions concentrated. In a preferred embodiment, the supernatant is administrated intramuscularly in a volume of 0.5 to 1 ml per injection, with two injections per week. In this embodiment a concentration of 30 million Wharton Jelly mesenchymal cells are grown on a plastic surface for approximately 24 hours. Supernatant is harvested, filter sterilized, and stored for administration. Other types of mesenchymal stem cells may be utilized, including mesenchymal stem cells derived bone marrow, peripheral blood, mobilized peripheral blood, endometrium, hair follicle, deciduous tooth, testicle, adipose tissue, skin, amniotic fluid, cord blood, omentum, muscle, amniotic membrane, periventricular fluid; and placental tissue.

In one aspect of the invention, potency of the conditioned media product may be quantified by use of assessing protein production. Such assays are well-known to one of skill in the art. Following the teachings of Jiao et al. [101], production of IL-10 may be quantified. For quantification of anti-inflammatory activity, the term “inflammation” will be understood by those skilled in the art to include any condition characterized by a localized or a systemic protective response, which may be elicited by physical trauma, infection, chronic diseases, such as those mentioned above, and/or chemical and/or physiological reactions to external stimuli (e.g., as part of an allergic response). Any such response, which may serve to destroy, dilute or sequester both the injurious agent and the injured tissue, may be manifested by, for example, heat, swelling, pain, redness, dilation of blood vessels and/or increased blood flow, invasion of the affected area by white blood cells, loss of function and/or any other symptoms known to be associated with inflammatory conditions. The term “inflammation” will thus also be understood to include any inflammatory disease, disorder or condition per se, any condition that has an inflammatory component associated with it, and/or any condition characterized by inflammation as a symptom, including, inter alia, acute, chronic, ulcerative, specific, allergic and necrotic inflammation, and other forms of inflammation known to those skilled in the art. The term thus also includes, for the purposes of this invention, inflammatory pain and/or fever caused by inflammation.

For quantification of effects that stem cells have on conditioned media, and therefore a quantification of the potency of conditioned media, one needs to first decide the therapeutic indication sought. If one seeks to utilize conditioned media for immune suppression, one may assess levels of immune modulatory components in said conditioned media. Examples of soluble immune suppressive factors include: IL-4 [102], IL-10 [103], IL-13 [104], TGF-b [105], soluble TNF-receptor [106], and IL-1 receptor agonist [107]. Membrane-bound immunoinhibitor molecules that may be shed by stem cells and therefore another marker for quantification of specific therapeutic properties: HLA-G [108], FasL [109], PD-1L [110], Decay Accelerating Factor [111], and membrane-associated TGF-b [112]. Enzymes whose biological activity causes alteration in supernatant composition to possess immune suppressive activities include indolamine 2,3 dioxygenase [113] and arginase type II [114]. In order to optimize desired immune suppressive ability, a wide variety of assays are known in the art, including mixed lymphocyte culture, ability to generate T regulatory cells in vitro, and ability to inhibit natural killer or CD8 cell cytotoxicity. In situations where increased angiogenic potential of said conditioned media therapeutic product is desired, assessment of proteins associated with stimulation of angiogenesis may be performed. These include VEGF[115], FGF1 [116], FGF2 [117], FGF4 [118], FrzA [119], and angiopoietin [120]. In some situations the cells in contact with media that generate conditioned media may be transfected with genes to allow for enhanced cellular viability, anti-apoptotic genes suitable for transfection may include bc1-2 [121], bcl-xl [122], and members of the XIAP family [123]. Alternatively it may be desired to increase the proliferative lifespan of said mesenchymal stem cells through transfection with enzymes associated with anti-senescence activity. Said enzymes may include telomerase or histone deacetylases.

In one embodiment mesenchymal cells are generated through culture and subsequently culture media is used for generation of a therapeutic composition. Said therapeutic composition is preferably generated in a medium that is free from human or animal products, with said medium also lacking phenol red. For extraction and growth of mesenchymal stem cells, the skilled practitioner of the invention is referred to examples known in the literature, which include U.S. Pat. No. 5,486,359 describing methods for culturing such and expanding mesenchymal stem cells, as well as providing antibodies for use in detection and isolation. Additionally, U.S. Pat. No. 5,942,225 teaches culture techniques and additives for differentiation of such stem cells which can be used in the context of the present invention to produce increased numbers of cells with ability to secrete agents that possess angiogenic activities. Although U.S. Pat. No. 6,387,369 teaches use of mesenchymal stem cells for regeneration of cardiac tissue, we believe that in accordance with published literature [124, 125] stem cells generated through these means are actually angiogenically potent and therefore may be utilized in the context of the current invention. Without being bound to a specific theory or mechanism of action, it appears that mesenchymal stem cells induce angiogenesis through production of factors such as vascular endothelial growth factor, hepatocyte growth factor, adrenomedullin, and insulin-like growth factor-1 [126], quantification of said growth factors may be useful in standardizing doses in the preparation of said stem cell conditioned media therapeutic product.

Historically, MSC are obtained from bone marrow sources for clinical use, although this source may have disadvantages because of the invasiveness of the donation procedure and the reported decline in number of bone marrow derived mesenchymal stem cells during aging. Alternative sources of mesenchymal stem cells include adipose tissue [127], placenta and Wharton's Jelly [128, 129], scalp tissue [130] and cord blood [131]. While mesenchymal stem cells generated from bone marrow, cord blood, and adipose tissue appear to possess similar morphology and phenotype, ability to induce colony formation appears to be highest using stem cells from adipose tissue and interestingly in contrast to bone marrow and adipose derived mesenchymal cells, only the cord blood derived cells lacked ability to undergo adipocyte differentiation. Within the context of the current invention, our data suggests that conditioned media generated using Wharton's Jelly as a source of cells possesses unique characteristics in contrast to adipose-derived stem cells. It is also known that the proliferative potential appears to be the highest with cord blood mesenchymal stem cells which were capable of expansion to approximately 20 times, whereas cord blood cells expanded an average of 8 times and bone marrow derived cells expanded 5 times [132]. Accordingly, one skilled in the art will understand that mesenchymal stem cells for use with the present invention may be selected upon individual patient characteristics and the end result sought.

In one embodiment, the treatment of immunological diseases is performed by administration of the stem cell conditioned media directly to its site of therapeutic activity, which in the case of many immune diseases is in the lymph nodes. For example, the therapeutic agent may be injected directly into the lymph nodes. Preferred lymph nodes for intranodal injections of inhibitors of T cell-dependent activation are the major lymph nodes located in the regions of the groin, the underarm and the neck. In another embodiment, the therapeutic agent is administered distal to the site of its therapeutic activity.

### EXAMPLES

### Example 1

A randomized phase III trial is conducted to test the safety and efficacy of perilympatically administered umbilical cord mesenchymal stem cells in patients with interferon resistant relapse remitting multiple sclerosis. Safety will be defined as freedom from treatment associated adverse events. Efficacy parameters, which will be assessed at weeks 12 and 52 will comprise endpoints of EDSS, expanded EDSS (Rating Neurologic Impairment in Multiple Sclerosis), the Scripps neurological rating scale (NRS), paced auditory serial addition test (PASAT), the nine-hole peg test, 25-foot walking time, short-form 36 (SF-36) quality of life questionnaire and gadolinium enhanced MRI scans of the brain and cervical spinal cord.

Umbilical cord mesenchymal stem cells are generated from the Wharton's Jelly (WJ-MSC). These are isolated from healthy mothers, aged 18-30, who have given birth to a healthy term fetus and no genetic family history, no cancer history, no hepatitis B virus (HBV), hepatitis C virus (HCV), human immunodeficiency virus (HIV), Epstein-Barr virus (EBV), cytomegalovirus (CMV) and syphilis in serum. The preparation of WJ-MSCs is performed in the laminar flow laboratory. Briefly, the umbilical cord is washed with phosphate buffered saline (PBS) twice and then dissected with scissors into pieces approximately 1 cm3 in volume. These tissue pieces are plated in a cell culture dish (Corning) in low-DMEM medium supplemented with 5% non-animal-derived serum. Cell cultures are maintained in a humidified atmosphere with 5% CO2 at 37° C. After 3 days of culture, the medium is replaced to remove the tissue and non-adherent cells, and changed twice weekly thereafter. Once 80% confluence is reached, the adherent cells (passage 0) are detached with 0.125% trypsin and passaged in the cell culture dish. The WJ-MSCs are cultured and expanded in laminar flow laboratory for 4 passages to prepare final cell products which should be sterile and all qualified for the examinations including aerobe, mycoplasma, HBV, HCV, HIV, EBV, CMV, syphilis, and endotoxin testing. Cells are administered at passage 5 and verified for expression of >90% CD90 and CD105 and <10% expression of CD14 and CD34.

Patients are administered the cells once every two days at a concentration of 1 million cells per injection in the perilymphatic area for a period of 2 weeks.

At weeks 12 and 52 after administration, patients receiving cells undergo an improvement in clinical parameters including EDSS, expanded EDSS (Rating Neurologic Impairment in Multiple Sclerosis), the Scripps neurological rating scale (NRS), paced auditory serial addition test (PASAT), the nine-hole peg test, 25-foot walking time, short-form 36 (SF-36) quality of life questionnaire and gadolinium enhanced MRI scans of the brain and cervical spinal cord.

### Example 2

A 40 y.o. woman diagnosed with secondary progressive multiple sclerosis in 2008 who was previously diagnosed with relapsing-remitting in 2003 presented for treatment in 2013 at an outpatient medical clinic. The patient had previously received treatment with Copaxone, Novatrone, Tysabri, Solumedrol, intravenous autologous stromal vascular fraction, and multiple intravenous human umbilical cord mesenchymal cell treatments. Symptoms at that time were inbalance, numbness, bladder incontinence, and impairment of eyesight in right.

Under compassionate use, the patient received injections of WJ-MSCs prepared in a manner consistent with example 1. 3 million cells were injected perilymphatically over each inguinal lymph bed. This treatment was repeated 2 days later. For a total of 12 million cells injected perilymphatically. In addition she receive two intravenous infusions of WJ-MSCs prepared in a manner consistent with example 1. Surprisingly, within hours of the perilymphatic injections the patient reported improvement in her balance, eyesight in the right eye, balance and bladder intolerance. The improvement has persisted for one month.

### REFERENCES

1. Orlic, D., et al., Bone marrow cells regenerate infarcted myocardium. Nature, 2001. 410(6829): p. 701-5.
2. Hamano, K., et al., Local implantation of autologous bone marrow cells for therapeutic angiogenesis in patients with ischemic heart disease: clinical trial and preliminary results. Jpn Circ J, 2001. 65(9): p. 845-7.
3. Stamm, C., et al., Autologous bone-marrow stem-cell transplantation for myocardial regeneration. Lancet, 2003. 361(9351): p. 45-6.
4. Kondziolka, D., et al., Neurotransplantation for patients with subcortical motor stroke: a phase 2 randomized trial. J Neurosurg, 2005. 103(1): p. 38-45.
5. Kordella, T., The Edmonton Protocol. The future of islet transplantation? Diabetes Forecast, 2003. 56(2): p. 58-62.
6. Shapiro, A. M., et al., International trial of the Edmonton protocol for islet transplantation. N Engl J Med, 2006. 355(13): p. 1318-30.
7. Bang, O. Y., et al., Autologous mesenchymal stem cell transplantation in stroke patients. Ann Neurol, 2005. 57(6): p. 874-82.
8. Mohamadnejad, M., et al., Phase 1 human trial of autologous bone marrow-hematopoietic stem cell transplantation in patients with decompensated cirrhosis. World J Gastroenterol, 2007. 13(24): p. 3359-63.
9. Wang, X. X., et al., Transplantation of autologous endothelial progenitor cells may be beneficial in patients with idiopathic pulmonary arterial hypertension: a pilot randomized controlled trial. J Am Coll Cardiol, 2007. 49(14): p. 1566-71.
10. Nizankowski, R., et al., The treatment of advanced chronic lower limb ischaemia with marrow stem cell autotransplantation. Kardiol Pol, 2005. 63(4): p. 351-60; discussion 361.
11. Billingham, R. E., L. Brent, and P. B. Medawar, Actively acquired tolerance of foreign cells. Nature, 1953. 172(4379): p. 603-6.
12. Dutta, P. and W. J. Burlingham, Tolerance to noninherited maternal antigens in mice and humans. Curr Opin Organ Transplant, 2009. 14(4): p. 439-47.
13. Clark, D. A., Is there any evidence for immunologically mediated or immunologically modifiable early pregnancy failure? J Assist Reprod Genet, 2003. 20(2): p. 63-72.
14. Ney, J. T., et al., Autochthonous liver tumors induce systemic T cell tolerance associated with T cell receptor down-modulation. Hepatology, 2009. 49(2): p. 471-81.
15. Cheung, A. F., et al., Regulated expression of a tumor-associated antigen reveals multiple levels of T-cell tolerance in a mouse model of lung cancer. Cancer Res, 2008. 68(22): p. 9459-68.
16. Bai, A., et al., Rapid tolerization of virus-activated tumor-specific CD8+ T cells in prostate tumors of TRAMP mice. Proc Natl Acad Sci USA, 2008. 105(35): p. 13003-8.
17. Whiteside, T. L., Down-regulation of zeta-chain expression in T cells: a biomarker of prognosis in cancer? Cancer Immunol Immunother, 2004. 53(10): p. 865-78.
18. Whiteside, T. L., Signaling defects in T lymphocytes of patients with malignancy. Cancer Immunol Immunother, 1999. 48(7): p. 346-52.
19. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.
20. Park, K. S., et al., Type II collagen oral tolerance; mechanism and role in collagen-induced arthritis and rheumatoid arthritis. Mod Rheumatol, 2009.
21. Womer, K. L., et al., A pilot study on the immunological effects of oral administration of donor major histocompatibility complex class II peptides in renal transplant recipients. Clin Transplant, 2008. 22(6): p. 754-9.
22. Faria, A. M. and H. L. Weiner, Oral tolerance: therapeutic implications for autoimmune diseases. Clin Dev Immunol, 2006. 13(2-4): p. 143-57.
23. Thompson, H. S., et al., Suppression of collagen induced arthritis by oral administration of type II collagen: changes in immune and arthritic responses mediated by active peripheral suppression. Autoimmunity, 1993. 16(3): p. 189-99.
24. Song, F., et al., The thymus plays a role in oral tolerance induction in experimental autoimmune encephalomyelitis. Ann N Y Acad Sci, 2004. 1029: p. 402-4.
25. Hanninen, A. and L. C. Harrison, Mucosal tolerance to prevent type 1 diabetes: can the outcome be improved in humans? Rev Diabet Stud, 2004. 1(3): p. 113-21.
26. Streilein, J. W. and J. Y. Niederkorn, Characterization of the suppressor cell(s) responsible for anterior chamber-associated immune deviation (ACAID) induced in BALB/c mice by P815 cells. J Immunol, 1985. 134(3): p. 1381-7.
27. Katagiri, K., et al., Using tolerance induced via the anterior chamber of the eye to inhibit Th2-dependent pulmonary pathology. J Immunol, 2002. 169(1): p. 84-9.
28. Benito-Leon, J., et al., A review about the impact of multiple sclerosis on health-related quality of life. Disabil Rehabil, 2003. 25(23): p. 1291-303.
29. Namaka, M., et al., Multiple sclerosis: etiology and treatment strategies. Consult Pharm, 2008. 23(11): p. 886-96.
30. Pilz, G., et al., Modern multiple sclerosis treatment—what is approved, what is on the horizon. Drug Discov Today, 2008. 13(23-24): p. 1013-25.
31. Loftus, B., et al., Autologous attenuated T-cell vaccine (Tovaxin) dose escalation in multiple sclerosis relapsing-remitting and secondary progressive patients nonresponsive to approved immunomodulatory therapies. Clin Immunol, 2009. 131(2): p. 202-15.
32. Spack, E. G., Antigen-specific therapies for the treatment of multiple sclerosis: a clinical trial update. Expert Opin Investig Drugs, 1997. 6(11): p. 1715-27.
33. Garren, H., et al., Phase 2 trial of a DNA vaccine encoding myelin basic protein for multiple sclerosis. Ann Neurol, 2008. 63(5): p. 611-20.
34. Darlington, C., MBP-8298, a synthetic peptide analog of myelin basic protein for the treatment of multiple sclerosis. Curr Opin Mol Ther, 2007. 9(4): p. 398-402.
35. Burt, R. K., et al., Autologous non-myeloablative haemopoietic stem cell transplantation in relapsing-remitting multiple sclerosis: a phase I/II study. Lancet Neurol, 2009. 8(3): p. 244-53.
36. Carreras, E., et al., CD34+ selected autologous peripheral blood stem cell transplantation for multiple sclerosis: report of toxicity and treatment results at one year of follow-up in 15 patients. Haematologica, 2003. 88(3): p. 306-14.
37. Fassas, A., et al., Hematopoietic stem cell transplantation for multiple sclerosis. A retrospective multicenter study. J Neurol, 2002. 249(8): p. 1088-97.
38. Bai, L., et al., Human bone marrow-derived mesenchymal stem cells induce Th2-polarized immune response and promote endogenous repair in animal models of multiple sclerosis. Glia, 2009.
39. Gordon, D., et al., Human mesenchymal stem cells abrogate experimental allergic encephalomyelitis after intraperitoneal injection, and with sparse CNS infiltration. Neurosci Lett, 2008. 448(1): p. 71-3.
40. Kassis, I., et al., Neuroprotection and immunomodulation with mesenchymal stem cells in chronic experimental autoimmune encephalomyelitis. Arch Neurol, 2008. 65(6): p. 753-61.
41. Zappia, E., et al., Mesenchymal stem cells ameliorate experimental autoimmune encephalomyelitis inducing T-cell anergy. Blood, 2005. 106(5): p. 1755-61.
42. Liang, J., et al., Allogeneic mesenchymal stem cells transplantation in treatment of multiple sclerosis. Mult Scler, 2009. 15(5): p. 644-6.
43. Mohyeddin Bonab, M., et al., Does mesenchymal stem cell therapy help multiple sclerosis patients? Report of a pilot study. Iran J Immunol, 2007. 4(1): p. 50-7.
44. Abeles, A. M. and M. H. Pillinger, The role of the synovial fibroblast in rheumatoid arthritis: cartilage destruction and the regulation of matrix metalloproteinases. Bulletin of the NYU hospital for joint diseases, 2006. 64(1-2): p. 20-4.
45. Smeets, T. J., et al., Analysis of the cell infiltrate and expression of proinflammatory cytokines and matrix metalloproteinases in arthroscopic synovial biopsies: comparison with synovial samples from patients with end stage, destructive rheumatoid arthritis. Annals of the rheumatic diseases, 2003. 62(7): p. 635-8.
46. Smeets, T. J., et al., Analysis of the cell infiltrate and expression of matrix metalloproteinases and granzyme B in paired synovial biopsy specimens from the cartilage-pannus junction in patients with RA. Annals of the rheumatic diseases, 2001. 60(6): p. 561-5.
47. Cope, A. P., H. Schulze-Koops, and M. Aringer, The central role of T cells in rheumatoid arthritis. Clinical and experimental rheumatology, 2007. 25(5 Suppl 46): p. S4-11.
48. Yamada, H., et al., Th1 but not Th17 cells predominate in the joints of patients with rheumatoid arthritis. Annals of the rheumatic diseases, 2008. 67(9): p. 1299-304.
49. Yamada, H., et al., Preferential accumulation of activated Th1 cells not only in rheumatoid arthritis but also in osteoarthritis joints. The Journal of rheumatology, 2011. 38(8): p. 1569-75.
50. Zizzo, G., et al., Synovial fluid-derived T helper 17 cells correlate with inflammatory activity in arthritis, irrespectively of diagnosis. Clinical immunology, 2011. 138(1): p. 107-16.
51. van Hamburg, J. P., et al., Th17 cells, but not Th1 cells, from patients with early rheumatoid arthritis are potent inducers of matrix metalloproteinases and proinflammatory cytokines upon synovial fibroblast interaction, including autocrine interleukin-17A production. Arthritis and rheumatism, 2011. 63(1): p. 73-83.
52. Church, L. D., et al., Rheumatoid synovial fluid interleukin-17-producing CD4 T cells have abundant tumor necrosis factor-alpha co-expression, but little interleukin-22 and interleukin-23R expression. Arthritis research & therapy, 2010. 12(5): p. R184.
53. Gullick, N. J., et al., Linking power Doppler ultrasound to the presence of the17 cells in the rheumatoid arthritis joint. PloS one, 2010. 5(9).
54. Chen, G., et al., Role of osteopontin in synovial Th17 differentiation in rheumatoid arthritis. Arthritis and rheumatism, 2010. 62(10): p. 2900-8.
55. Evans, H. G., et al., In vivo activated monocytes from the site of inflammation in humans specifically promote Th17 responses. Proceedings of the National Academy of Sciences of the United States of America, 2009. 106(15): p. 6232-7.
56. Pelletier, M., et al., Evidence for a cross-talk between human neutrophils and Th17 cells. Blood, 2010. 115(2): p. 335-43.
57. Choosing Medications for Rheumatoid Arthritis: Clinician's Guide, in Comparative Effectiveness Review Summary Guides for Clinicians. 2007: Rockville (MD).
58. Segerer, S. E., et al., The glycoprotein-hormones activin A and inhibin A interfere with dendritic cell maturation. Reprod Biol Endocrinol, 2008. 6: p. 17.
59. Segerer, S. E., et al., Impact of female sex hormones on the maturation and function of human dendritic cells. Am J Reprod Immunol, 2009. 62(3): p. 165-73.
60. Shojaeian, J., et al., Immunosuppressive effect of pregnant mouse serum on allostimulatory activity of dendritic cells. J Reprod Immunol, 2007. 75(1): p. 23-31.
61. Zarnani, A. H., et al., Microenvironment of the feto-maternal interface protects the semiallogenic fetus through its immunomodulatory activity on dendritic cells. Feral Steril, 2008. 90(3): p. 781-8.
62. Jurgens, B., et al., Interferon-gamma-triggered indoleamine 2,3-dioxygenase competence in human monocyte-derived dendritic cells induces regulatory activity in allogeneic T cells. Blood, 2009. 114(15): p. 3235-43.
63. Brenk, M., et al., Tryptophan deprivation induces inhibitory receptors ILT3 and ILT4 on dendritic cells favoring the induction of human CD4+ CD25+ Foxp3+ T regulatory cells. J Immunol, 2009. 183(1): p. 145-54.
64. Mellor, A. L., et al., Prevention of T cell-driven complement activation and inflammation by tryptophan catabolism during pregnancy. Nat Immunol, 2001. 2(1): p. 64-8.
65. Lee, J. R., et al., Pattern of recruitment of immunoregulatory antigen-presenting cells in malignant melanoma. Lab Invest, 2003. 83(10): p. 1457-66.
66. Botella-Estrada, R., et al., Cytokine expression and dendritic cell density in melanoma sentinel nodes. Melanoma Res, 2005. 15(2): p. 99-106.
67. Curiel, T. J., et al., Blockade of B7-H1 improves myeloid dendritic cell-mediated antitumor immunity. Nat Med, 2003. 9(5): p. 562-7.
68. Almand, B., et al., Clinical significance of defective dendritic cell differentiation in cancer. Clin Cancer Res, 2000. 6(5): p. 1755-66.
69. Almand, B., et al., Increased production of immature myeloid cells in cancer patients: a mechanism of immunosuppression in cancer. J Immunol, 2001. 166(1): p. 678-89.
70. Zheng, X., et al., Reinstalling antitumor immunity by inhibiting tumor-derived immunosuppressive molecule IDO through RNA interference. J Immunol, 2006. 177(8): p. 5639-46.
71. Johnson, B., et al., Physiology and therapeutics of vascular endothelial growth factor in tumor immunosuppression. Curr Mol Med, 2009. 9(6): p. 702-7.
72. Osada, T., et al., The effect of anti-VEGF therapy on immature myeloid cell and dendritic cells in cancer patients. Cancer Immunol Immunother, 2008. 57(8): p. 1115-24.
73. Min, S. Y., et al., Antigen-induced, tolerogenic CD11c+,CD11b+ dendritic cells are abundant in Peyer's patches during the induction of oral tolerance to type II collagen and suppress experimental collagen-induced arthritis. Arthritis Rheum, 2006. 54(3): p. 887-98.
74. Park, M. J., et al., Indoleamine 2,3-dioxygenase-expressing dendritic cells are involved in the generation of CD4+ CD25+ regulatory T cells in Peyer's patches in an orally tolerized, collagen-induced arthritis mouse model. Arthritis Res Ther, 2008. 10(1): p. R11.
75. Viney, J. L., et al., Expanding dendritic cells in vivo enhances the induction of oral tolerance. J Immunol, 1998. 160(12): p. 5815-25.
76. Shiokawa, A., et al., IL-10 and IL-27 producing dendritic cells capable of enhancing IL-10 production of T cells are induced in oral tolerance. Immunol Lett, 2009. 125(1): p. 7-14.
77. Stein-Streilein, J. and C. Watte, Cross talk among cells promoting anterior chamber-associated immune deviation. Chem Immunol Allergy, 2007. 92: p. 115-30.
78. Zhang, H., et al., Involvement of Foxp3-expressing CD4+ CD25+ regulatory T cells in the development of tolerance induced by transforming growth factor-beta2-treated antigen-presenting cells. Immunology, 2008. 124(3): p. 304-14.
79. Min, W. P., et al., Synergistic tolerance induced by LF15-0195 and anti-CD45RB monoclonal antibody through suppressive dendritic cells. Transplantation, 2003. 75(8): p. 1160-5.
80. Min, W. P., et al., Inhibitory feedback loop between tolerogenic dendritic cells and regularoty T cells in transplant tolerance. J Immunol, 2003. 170: p. 1304-1312.
81. Yang, J., et al., LF15-0195 generates tolerogenic dendritic cells by suppression of NF-kappaB signaling through inhibition of IKK activity. J Leukoc Biol, 2003. 74(3): p. 438-47.
82. Min, W. P., et al., Dendritic cells genetically engineered to express Fas ligand induce donor-specific hyporesponsiveness and prolong allograft survival. J Immunol, 2000. 164(1): p. 161-7.
83. Gainer, A. L., et al., Improved survival of biolistically transfected mouse islet allografts expressing CTLA4-Ig or soluble Fas ligand. Transplantation, 1998. 66(2): p. 194-9.
84. Ichim, T. E., et al., RNA interference: a potent tool for gene-specific therapeutics. Am J Transplant, 2004. 4(8): p. 1227-36.
85. Ichim, T. E., R. Zhong, and W. P. Min, Prevention of allograft rejection by in vitro generated tolerogenic dendritic cells. Transpl Immunol, 2003. 11(3-4): p. 295-306.
86. Hill, J. A., et al., Immune modulation by silencing IL-12 production in dendritic cells using small interfering RNA. J Immunol, 2003. 171(2): p. 691-6.
87. L1, M., et al., Induction of RNA interference in dendritic cells. Immunol Res, 2004. 30(2): p. 215-30.
88. Min, W. P., et al., Inhibitory feedback loop between tolerogenic dendritic cells and regulatory T cells in transplant tolerance. J Immunol, 2003. 170(3): p. 1304-12.
89. Germain, R. N., Special regulatory T-cell review: A rose by any other name: from suppressor T cells to Tregs, approbation to unbridled enthusiasm. Immunology, 2008. 123(1): p. 20-7.
90. Hall, B. M., et al., Specific unresponsiveness in rats with prolonged cardiac allograft survival after treatment with cyclosporine. III. Further characterization of the CD4+ suppressor cell and its mechanisms of action. J Exp Med, 1990. 171(1): p. 141-57.
91. Pearce, N. W., et al., Specific unresponsiveness in rats with prolonged cardiac allograft survival after treatment with cyclosporine. V. Dependence of CD4+ suppressor cells on the presence of alloantigen and cytokines, including interleukin 2. Transplantation, 1993. 55(2): p. 374-80.
92. Sakaguchi, S., et al., Immunologic self-tolerance maintained by activated T cells expressing IL-2 receptor alpha-chains (CD25). Breakdown of a single mechanism of self-tolerance causes various autoimmune diseases. J Immunol, 1995. 155(3): p. 1151-64.
93. Ptak, W. and R. K. Gershon, Immunological agnosis: a state that derives from T suppressor cell inhibition of antigen-presenting cells. Proc Natl Acad Sci USA, 1982. 79(8): p. 2645-8.
94. Randolph, G. J., V. Angeli, and M. A. Swartz, Dendritic-cell trafficking to lymph nodes through lymphatic vessels. Nat Rev Immunol, 2005. 5(8): p. 617-28.
95. Baek, S. J., S. K. Kang, and J. C. Ra, In vitro migration capacity of human adipose tissue-derived mesenchymal stem cells reflects their expression of receptors for chemokines and growth factors. Exp Mol Med, 2011. 43(10): p. 596-603.
96. Liu, H., et al., Hypoxic preconditioning advances CXCR4 and CXCR7 expression by activating HIF-1alpha in MSCs. Biochem Biophys Res Commun, 2010. 401(4): p. 509-15.
97. Faure-Andre, G., et al., Regulation of dendritic cell migration by CD74, the MHC class II-associated invariant chain. Science, 2008. 322(5908): p. 1705-10.
98. Cera, M. R., et al., Increased DC trafficking to lymph nodes and contact hypersensitivity in junctional adhesion molecule-A-deficient mice. J Clin Invest, 2004. 114(5): p. 729-38.
99. Yen, J. H., T. Khayrullina, and D. Ganea, PGE2-induced metalloproteinase-9 is essential for dendritic cell migration. Blood, 2008. 111(1): p. 260-70.
100. Ratzinger, G., et al., Matrix metalloproteinases 9 and 2 are necessary for the migration of Langerhans cells and dermal dendritic cells from human and murine skin. J Immunol, 2002. 168(9): p. 4361-71.
101. Jiao, J., et al., A mesenchymal stem cell potency assay. Methods in molecular biology, 2011. 677: p. 221-31.
102. Jansen, J. H., et al., Interleukin-4. A regulatory protein. Blut, 1990. 60(5): p. 269-74.
103. Zhou, X., et al., Boosting interleukin-10 production: therapeutic effects and mechanisms. Curr Drug Targets Immune Endocr Metabol Disord, 2005. 5(4): p. 465-75.
104. Mentink-Kane, M. M. and T. A. Wynn, Opposing roles for IL-13 and IL-13 receptor alpha 2 in health and disease. Immunol Rev, 2004. 202: p. 191-202.
105. Kriegel, M. A., et al., Transforming growth factor-beta: recent advances on its role in immune tolerance. Curr Rheumatol Rep, 2006. 8(2): p. 138-44.
106. Fernandez-Botran, R., F. A. Crespo, and X. Sun, Soluble cytokine receptors in biological therapy. Expert Opin Biol Ther, 2002. 2(6): p. 585-605.
107. Dayer, J. M., Evidence for the biological modulation of IL-1 activity: the role of IL-1Ra. Clin Exp Rheumatol, 2002. 20(5 Suppl 27): p. S14-20.
108. Rouas-Freiss, N., et al., HLA-G proteins in cancer: do they provide tumor cells with an escape mechanism? Cancer Res, 2005. 65(22): p. 10139-44.
109. Bohana-Kashtan, O. and C. I. Civin, Fas ligand as a tool for immunosuppression and generation of immune tolerance. Stem Cells, 2004. 22(6): p. 908-24.
110. Okazaki, T. and T. Honjo, The PD-1-PD-L pathway in immunological tolerance. Trends Immunol, 2006. 27(4): p. 195-201.
111. Longhi, M. P., et al., Holding T cells in check—a new role for complement regulators? Trends Immunol, 2006. 27(2): p. 102-8.
112. Wahl, S. M., J. M. Orenstein, and W. Chen, TGF-beta influences the life and death decisions of T lymphocytes. Cytokine Growth Factor Rev, 2000. 11(1-2): p. 71-9.
113. Mellor, A. L. and D. H. Munn, IDO expression by dendritic cells: tolerance and tryptophan catabolism. Nat Rev Immunol, 2004. 4(10): p. 762-74.
114. Serafini, P., I. Borrello, and V. Bronte, Myeloid suppressor cells in cancer: recruitment, phenotype, properties, and mechanisms of immune suppression. Semin Cancer Biol, 2006. 16(1): p. 53-65.
115. Matsumoto, R., et al., Vascular endothelial growth factor-expressing mesenchymal stem cell transplantation for the treatment of acute myocardial infarction. Arterioscler Thromb Vasc Biol, 2005. 25(6): p. 1168-73.
116. Klein, S., M. Roghani, and D. B. Rifkin, Fibroblast growth factors as angiogenesis factors: new insights into their mechanism of action. Exs, 1997. 79: p. 159-92.
117. Chen, C. H., et al., Fibroblast growth factor 2: from laboratory evidence to clinical application. Curr Vasc Pharmacol, 2004. 2(1): p. 33-43.
118. Grines, C., et al., Angiogenic gene therapy with adenovirus 5 fibroblast growth factor-4 (Ad5FGF-4): a new option for the treatment of coronary artery disease. Am J Cardiol, 2003. 92(9B): p. 24N-31N.
119. Dufourcq, P., et al., FrzA, a secreted frizzled related protein, induced angiogenic response. Circulation, 2002. 106(24): p. 3097-103.
120. Morisada, T., et al., Angiopoietins and angiopoietin-like proteins in angiogenesis. Endothelium, 2006. 13(2): p. 71-9.
121. Murphy, E., K. Imahashi, and C. Steenbergen, Bc1-2 regulation of mitochondrial energetics. Trends Cardiovasc Med, 2005. 15(8): p. 283-90.
122. Harada, H. and S. Grant, Apoptosis regulators. Rev Clin Exp Hematol, 2003. 7(2): p. 117-38.
123. Guegan, C., et al., PTD-XIAP protects against cerebral ischemia by anti-apoptotic and transcriptional regulatory mechanisms. Neurobiol Dis, 2006. 22(1): p. 177-86.
124. Caplan, A. I. and J. E. Dennis, Mesenchymal stem cells as trophic mediators. J Cell Biochem, 2006.
125. Shyu, K. G., et al., Mesenchymal stem cells are superior to angiogenic growth factor genes for improving myocardial performance in the mouse model of acute myocardial infarction. J Biomed Sci, 2006. 13(1): p. 47-58.
126. Nagaya, N., et al., Transplantation of mesenchymal stem cells improves cardiac function in a rat model of dilated cardiomyopathy. Circulation, 2005. 112(8): p. 1128-35.
127. Knippenberg, M., et al., Adipose tissue-derived mesenchymal stem cells acquire bone cell-like responsiveness to fluid shear stress on osteogenic stimulation. Tissue Eng, 2005. 11(11-12): p. 1780-8.
128. Portmann-Lanz, C. B., et al., Placental mesenchymal stem cells as potential autologous graft for pre- and perinatal neuroregeneration. Am J Obstet Gynecol, 2006. 194(3): p. 664-73.
129. Zhang, X., et al., Mesenchymal progenitor cells derived from chorionic villi of human placenta for cartilage tissue engineering. Biochem Biophys Res Commun, 2006. 340(3): p. 944-52.
130. Shih, D. T., et al., Isolation and characterization of neurogenic mesenchymal stem cells in human scalp tissue. Stem Cells, 2005. 23(7): p. 1012-20.
131. Kadivar, M., et al., In vitro cardiomyogenic potential of human umbilical vein-derived mesenchymal stem cells. Biochem Biophys Res Commun, 2006. 340(2): p. 639-47.
132. Kern, S., et al., Comparative Analysis of Mesenchymal Stem Cells from Bone Marrow, Umbilical Cord Blood or Adipose Tissue. Stem Cells, 2006.

What is claimed is:

1. A method of immune modulating a mammal comprising identifying a mammal in need of immune modulation and administering an immune modulatory cell perilymphatically or intralymphatically into said mammal.

2. The method of claim 1, wherein said immune modulatory cell is selected from a group of cells comprising of: a) mesenchymal stem cells; b) T regulatory cells; c) type 2 monocytes; d) CD5 positive B cells; e) type 2 NKT cells; f) tolerogenic dendritic cells; g) gamma delta T cells; h) T cells with immune regulatory properties; i) CD34 cells; j) very small embryonic like stem cells and k) Sertoli cells.

3. The method of claim 2, wherein said mesenchymal stem cell is derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

4. The method of claim 3, wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

5. The method of claim 4, wherein said mesenchymal stem cells do not express substantial levels of HLA-DR, CD117, and CD45.

6. The method of claim 3, wherein said mesenchymal stem cells are generated from a pluripotent stem cell.

7. The method of claim 6, wherein said pluripotent stem cell is selected from a group comprising of: a) an embryonic stem cell; b) an inducible pluripotent stem cell; c) a parthenogenic stem cell; and d) a somatic cell nuclear transfer derived stem cell.

8. The method of claim 7, wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

9. The method of claim 7, wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.

10. The method of claim 7, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

11. The method of claim 7, wherein said somatic cell nuclear transfer derived stem cells possess a phenotype negative for SSEA-1 and positive for SSEA-3, SSEA-4, TRA-1-60, TRA-1-81, and alkaline phosphatase.

12. The method of claim 6, wherein said mesenchymal stem cells are differentiated from a pluripotent stem cell source through culture in the presence of an inhibitor of the SMAD-2/3 pathway.

13. The method of claim 12, wherein said mesenchymal stem cells are differentiated from a pluripotent stem cell source through culture in the presence of an inhibitor nucleic acid targeting the SMAD-2/3 pathway.

14. The method of claim 13, wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme.

15. The method of claim 13, wherein said inhibitor of the SMAD-2/3 pathway is a small molecule inhibitor.

16. The method of claim 15, wherein said small molecule inhibitor is SB-431542.

17. The method of claim 6, wherein a selection process is used to enrich for mesenchymal stem cells differentiated from said pluripotent stem cell population.

18. The method of claim 17, wherein said enrichment method comprises of positively selecting for cells expressing a marker associated with mesenchymal stem cells.

19. The method of claim 18, wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

20. The method of claim 1, wherein said immune modulatory cells are autologous, allogeneic or xenogeneic to the recipient.
